(19) United States
(12) Patent Application Publication
(10) Pub. No.: US 2017/0306009 A1
(43) Pub. Date: Oct. 26, 2017

(54) METHODS FOR MODULATING THE GLYCOSYLATION PROFILE OF RECOMBINANT PROTEINS USING DISSOLVED OXYGEN

(71) Applicant: AbbVie Inc., North Chicago, IL (US)

(72) Inventor: Xiaobei Zeng, Carolina, PR (US)

(21) Appl. No.: 15/479,709

(22) Filed: Apr. 5, 2017

Related U.S. Application Data
(63) Continuation of application No. 14/813,561, filed on Jul. 30, 2015.
(60) Provisional application No. 62/033,412, filed on Aug. 5, 2014.

(51) Int. Cl. C07K 16/24; C12P 21/00; A61K 39/395; C07K 16/00

(52) U.S. Cl. CPC C07K 16/241; C07K 16/00; C12P 21/005; A61K 39/39591; C07K 2317/14; C07K 2317/41; C07K 2317/56; C07K 2317/21

(57) ABSTRACT

The present invention relates to the field of protein production, and in particular to methods and compositions for modulating glycosylation of recombinant proteins expressed in host cells by supplementing the production media with dissolved oxygen.

### RELATED APPLICATIONS

The present application is a continuation of U.S. patent application Ser. No. 14/813,561, filed on Jul. 30, 2015, which in turn, claims priority to U.S. Provisional Patent Application No. 62/033,412, filed on Aug. 5, 2014. The entire contents of each of the foregoing applications are incorporated herein by reference.

### BACKGROUND OF THE INVENTION

The instant invention relates to the field of recombinant protein (e.g., antibody or DVD-Ig) production, and, in particular, to methods and compositions for modulating the glycosylation profile and, thus, the heterogeneity of recombinant proteins expressed in host cells. The production of recombinant proteins for biopharmaceutical applications typically involves the use of cell cultures that are known to produce recombinant proteins exhibiting varying levels of heterogeneity. The basis for such heterogeneity includes, but is not limited to, the presence of distinct glycosylation profiles in the produced recombinant proteins. For example, but not by way of limitation, such heterogeneity can be observed as an increase or decrease in high mannose N-glycan and galactosylated N-glycan levels as well modulation of agalactosyl N-glycan levels.

Protein glycosylation is an important post-translational modification that has been found to have a significant impact on various aspects of protein structure and function (Rudd P. M. et al., (1997) Crit Rev Biochem Mol Biol, 32(1):1-100; Wright A. et al., (1997) Trends Biotechnol, 15(1):26-32; Wyss D. F. et al., (1996) Curr Opin Biotechnol, 1996. 7(4):409-416). Among the various types of post-translational modification, protein glycosylation has been shown to have a significant impact on the activity, binding, pharmacokinetics (PK) and immunogenicity of recombinant glycoprotein therapeutics (Elliott S. et al., (2003) Nat Biotechnol, 21(4):414-21; Kanda Y. et al., (2007) Glycobiology 17(1):104-118; Mori K. et al., (2004) Biotechnol Bioeng, 88(7):901-908; Perlman S. et al., (2003) J Clin Endocrinol Metab, 88(7): 3227-35; Shields R. L. et al., (2002) J Biol Chem. 277(30):26733-26740; Takeuchi M. et al., (1989) Proc Natl Acad Sci USA, 86(20):7819-7822; Weenen C. et al., (2004) J Clin Endocrinol Metab, 89(10):5204-5212; Coloma, M. J. et al., (1999) J Immunol, 162(4):2162-70; Davis J. et al., (2001) Biotechnol Bioeng, 74(4):288-294; Wallick S. C. et al., (1988) J Exp Med, 168(3):1099-1109; Fukuda M. N. et al., (1989) Blood, 73(1):84-89; Jones A. J. et al., (2007) Glycobiology, 17(5):529-540; Keck R. et al., (2008) Biologicals, 36(1):49-60; Stork R. et al., (2008) J Biol Chem, 283(12):7804-7812; Noguchi A. et al., (1995) J Biochem, 117(1):59-62; Rudd P. M. et al., (2001) Science, 291(5512):2370-2376; Sathyamoorthy N. et al., (1991) Mol Cell Biochem, 102(2):139-47). As a result, protein glycosylation, as a critical quality attribute, must be considered in the manufacture of recombinant proteins.

Protein glycosylation involves a spectrum of oligosaccharide structures (glycans) that are generally classified into 2 categories corresponding to the amino acids they are attached to. N-linked glycosylation includes the pattern of oligosaccharides that are attached to asparagine residues and are generally attached at a conserved consensus amino acid sequence (Asn-X-Ser/Thr). Serine/threonine (O-linked) glycosylation includes the pattern of oligosaccharides that are attached to serine/threonine residues and do not generally have a conserved sequence for attachment. Both N- and O-linked protein glycosylation occurs in the endoplasmic reticulum (ER) and Golgi apparatus. Inside these organelles the metabolic pathways are characterized by the step-wise addition and/or removal of individual monosaccharides, which when viewed from a macroscopic perspective, comprises a network of metabolic reactions. In general, the pathway is associated with the initial formation of high mannose glycans in the ER, which are further trimmed and processed by various glycosylation enzymes to facilitate the formation of agalactosyl glycans (e.g., G0, G0-GlcNAc, G0F, G0F-GlcNAc), and subsequently the more highly processed N-glycans comprising one galactose moiety (e.g., G1F, G1F-GlcNAc) or two galactose moieties (e.g., G2F). Subsequent reactions are possible upon the formation of G2F, namely the addition of sialic acid. In the present invention the highest extent of N-glycan processing is considered to be G2F since in the CHO expression system utilized, and the expressed protein evaluated, sialic acid levels are negligible.

The pattern of glycosylation reactions is ultimately what determines the glycosylation pattern (glycoform) observed on recombinant glycoprotein therapeutics. Individual differences in the monosaccharide composition of glycans attached to a particular glycosylation site on a protein is called microhetereogeneity. In the protein N- and O-glycosylation pathways there exists a variety of control steps that ultimately determine the final glycoform profile. Some of the control steps include the relative activity of the protein glycosylation enzymes, the relative levels of donor nucleotide-sugar substrates, the localization of the enzymes in the protein secretory pathway, as well as the innate substrate specificities for each of the enzymes involved. The various control points of the protein N-glycosylation pathway has been reviewed and simulated (Hossler P. et al., (2007) PLoS One 2(8):e713).

Due to the inherent variability demonstrated within the glycosylation pathway, the nature of the recombinant protein, as well as manufacturing process conditions used to produce the protein; numerous studies have identified particular glycoform profiles which are either beneficial or detrimental to the protein's physiochemical characteristics. For example, excess mannose has been shown to have immunomodulatory activities (Sathyamoorthy N. et al., (1991) Mol Cell Biochem, 102(2):139-147) and excess GlcNAc has been shown to lead to a reduction in the overall PK (Jones A. J. et al., (2007) Glycobiology, 17(5):529-540). Increased sialylation has been shown to facilitate a longer circulatory half-life (Elliott S. et al., (2003) Nat Biotechnol, 21(4):414-21). Decreased fucosylation has been shown to facilitate an increase in antibody dependent cellular cytotoxicity (ADCC) (Kanda Y. et al., (2007) Glycobiology, 17(1):104-18; Shields R. L. et al., (2002) J Biol Chem, 277(30):26733-26740). Having the capability to fine tune the protein glycosylation metabolic pathway facilitates the ability to control the resulting oligosaccharide profile of the therapeutic protein.

Accordingly, there is a need in the art for compositions and methods for the targeted modulation of protein glycosylation.

### SUMMARY OF THE INVENTION

The instant invention addresses this need by discovering that the addition of dissolved oxygen to cell culture media can have a profound impact on the glycosylation profile, e.g., mannosylated N-glycan species, of recombinant proteins.

Accordingly, in one aspect, the invention provides methods of producing a composition comprising a recombinant protein with a modulated glycosylation profile. The methods include culturing a host cell expressing the recombinant protein in cell culture media supplemented with dissolved oxygen, thereby producing the composition comprising the recombinant protein with a modulated glycosylation profile as compared to a control, wherein the control is a composition comprising a recombinant protein produced by culturing a host cell expressing the recombinant protein in cell culture media which is not supplemented with the dissolved oxygen.

In one embodiment, the methods further comprise purifying the composition comprising the recombinant protein with a modulated glycosylation profile.

In another embodiment, the recombinant protein is an antibody or antigen-binding portion thereof. In a particular embodiment, the antibody is an anti-TNFα antibody. In yet another embodiment, the anti-TNFα antibody is adalimumab, or an antigen binding fragment thereof. In yet another embodiment, the recombinant protein is a dual variable domain immunoglobulin (DVD-Ig). In one embodiment, the recombinant protein is selected from the group consisting of a TVD-Ig, a half-body and a RAB.

In one embodiment of the invention, the dissolved oxygen is selected from the group consisting of 10%, 20%, 30%, 40%, 50%, 60%, 70%, and 80%.

In one embodiment, the cell culture media is supplemented with a sufficient amount of the dissolved oxygen to achieve a dissolved oxygen concentration selected from the group consisting of about 10%, about 20%, about 30%, about 40%, about 50%, about 60%, about 70%, and about 80%. In a particular embodiment, the dissolved oxygen concentration is 10%. In one embodiment, the dissolved oxygen is 20%. In another embodiment, the dissolved oxygen is 30%. In yet another embodiment, the dissolved oxygen is 40%. In a particular embodiment, the dissolved oxygen is 50%. In one embodiment, the dissolved oxygen is 60%. In another embodiment, the dissolved oxygen is 70%. In yet another embodiment, the dissolved oxygen is 80%.

In one embodiment of the invention, the modulated glycosylation profile of the recombinant protein comprises modulation of a galactosylation level, a mannosylated N-glycan level or an agalactosyl N-glycan level in the recombinant protein.

In one embodiment of the invention, the modulated glycosylation profile of the recombinant protein comprises modulation of the galactosylation level in the recombinant protein.

In another embodiment of the invention, the modulation of the mannosylated N-glycan level comprises an increase in the mannosylation level of the recombinant protein, e.g., an increase in the level of Man 5 glycan, Man 6 glycan, Man 7 glycan, Man 8 glycan and/or Man 9 glycan. In a further embodiment, the increase in the level of Man 5 glycan, Man 6 glycan, Man 7 glycan, Man 8 glycan and/or Man 9 glycan is a increase of about 0.1%, 1%, 1.2%, 1.5%, 2%, 2.2%, 2.5%, 3%, 3.2%, 3.5%, 4%, 4.2%, 4.5%, 5%, 10%, 15%, 20%, or 25%.

In another embodiment of the invention, the modulation of the mannosylated N-glycan level comprises a decrease in the mannosylation level of the recombinant protein, e.g., a decrease in the level of Man 5 glycan, Man 6 glycan, Man 7 glycan, Man 8 glycan and/or Man 9 glycan. In a further embodiment, the decrease in the level of Man 5 glycan, Man 6 glycan, Man 7 glycan, Man 8 glycan and/or Man 9 glycan is a decrease of about 0.1%, 1%, 1.2%, 1.5%, 2%, 2.2%, 2.5%, 2.55%, 3%, 3.2%, 3.5%, 4%, 4.2%, 4.5%, 5%, 10%, 15%, 20%, or 25%. In yet another embodiment of the invention, the decrease in the level of Man 5 glycan, Man 6 glycan, Man 7 glycan, Man 8 glycan and/or Man 9 glycan is a decrease of about 2.55%.

In one embodiment of the invention, the modulated glycosylation profile of the recombinant protein comprises modulation of the agalactosyl N-glycan level in the recombinant protein.

In one embodiment, the host cell is a CHO cell.

In another aspect, the present invention provides methods of producing compositions comprising an antibody, or antigen binding fragment thereof, with a modulated glycosylation profile. The methods include culturing a host cell expressing the antibody, or antigen binding fragment thereof, in cell culture media supplemented with dissolved oxygen, thereby producing the composition comprising the antibody, or antigen binding fragment thereof, with an increased level of mannosylated N-glycans compared to a control, wherein the control is a composition comprising an antibody, or antigen binding fragment thereof, produced by culturing a host cell expressing the antibody, or antigen binding fragment thereof, in cell culture media which is not supplemented with dissolved oxygen. In a further embodiment, the antibody is adalimumab, or an antigen binding fragment thereof.

In another aspect, the present invention provides methods of producing compositions comprising an antibody, or antigen binding fragment thereof, with a modulated glycosylation profile. The methods include culturing a host cell expressing the antibody, or antigen binding fragment thereof, in cell culture media supplemented with dissolved oxygen, thereby producing the composition comprising the antibody, or antigen binding fragment thereof, with a decreased level of mannosylated N-glycans compared to a control, wherein the control is a composition comprising an antibody, or antigen binding fragment thereof, produced by culturing a host cell expressing the antibody, or antigen binding fragment thereof, in cell culture media which is not supplemented with dissolved oxygen. In a further embodiment, the antibody is adalimumab, or an antigen binding fragment thereof.

In another aspect, the present invention provides methods of producing compositions comprising an antibody, or antigen binding fragment thereof, with a modulated glycosylation profile. The methods include culturing a host cell expressing the antibody, or antigen binding fragment thereof, in cell culture media supplemented with dissolved oxygen, thereby producing the composition comprising the antibody, or antigen binding fragment thereof, with a 0.1-15% increase in the level of mannosylated N-glycans as compared to a control, wherein the control is a composition comprising an antibody, or antigen binding fragment thereof, produced by culturing a host cell expressing the antibody, or antigen binding fragment thereof, in cell culture media which is not supplemented with dissolved oxygen. In a further embodiment, the antibody is adalimumab, or an antigen binding fragment thereof.

In another aspect, the present invention provides methods of producing compositions comprising an antibody, or antigen binding fragment thereof, with a modulated glycosylation profile. The methods include culturing a host cell expressing the antibody, or antigen binding fragment thereof, in cell culture media supplemented with dissolved oxygen, thereby producing the composition comprising the antibody, or antigen binding fragment thereof, with a 0.1-15% decrease in the level of mannosylated N-glycans as compared to a control, wherein the control is a composition comprising an antibody, or antigen binding fragment thereof, produced by culturing a host cell expressing the antibody, or antigen binding fragment thereof, in cell culture media which is not supplemented with dissolved oxygen. In another embodiment, the decrease in the level of mannosylated N-glycans as compared to a control is a decrease of about 2.55%. In a further embodiment, the antibody is adalimumab, or an antigen binding fragment thereof. In one embodiment, the antibody, or antigen binding fragment thereof, further comprises a decreased level of mannosylated N-glycans as compared to the control.

In one aspect, the present invention provides compositions comprising a cell culture media comprising dissolved oxygen. In one embodiment the composition comprising a cell culture media comprises a dissolved oxygen concentration selected from the group consisting of about 10%, about 20%, about 30%, about 40%, about 50%, about 60%, about 70%, and about 80%.

In a further aspect, the present invention provides pharmaceutical compositions comprising compositions produced by the methods of the invention and a pharmaceutically acceptable carrier.

In one aspect, the present invention provides compositions comprising a therapeutic protein with a modulated glycosylation profile produced by the methods of the invention. In a particular embodiment, the therapeutic protein is an antibody.

In another aspect, the present invention provides compositions comprising adalimumab, wherein the adalimumab comprises a 0.1-15% increase in the level of mannosylated N-glycans as compared to a control, wherein the control is a composition comprising adalimumab produced by culturing a host cell expressing adalimumab in cell culture media which is not supplemented with dissolved oxygen.

In another aspect, the present invention provides compositions comprising adalimumab, wherein the adalimumab comprises a 0.1-15% decrease in the level of mannosylated N-glycans as compared to a control, wherein the control is a composition comprising adalimumab produced by culturing a host cell expressing adalimumab in cell culture media which is not supplemented with dissolved oxygen. In a further aspect, the decrease in the level of mannosylated N-glycans as compared to a control is a decrease of about 2.55%.

In yet another aspect, the present invention provides methods of producing compositions comprising adalimumab. The methods include culturing a host cell expressing adalimumab in cell culture media supplemented with dissolved oxygen, thereby producing the compositions comprising adalimumab. In one embodiment, the cell culture media comprises a dissolved oxygen concentration selected from the group consisting of about 10%, about 20%, about 30%, about 40%, about 50%, about 60%, about 70%, and about 80%.

In a further aspect, the present invention provides methods of modulating the glycosylation profile of adalimumab. The methods include, culturing a host cell expressing adalimumab in cell culture media supplemented with an amount of dissolved oxygen sufficient to modulate the glycosylation profile of adalimumab, thereby modulating the glycosylation profile of adalimumab. In one embodiment, the cell culture media comprises a dissolved oxygen concentration selected from the group consisting of about 10%, about 20%, about 30%, about 40%, about 50%, about 60%, about 70%, and about 80%.

In another aspect, the present invention provides compositions comprising an antibody comprising the heavy and light chain variable domains of adalimumab, wherein less than 5% of the total N-linked oligosaccharides present on said antibody are of a mannosylated N-glycan form. In one embodiment, the antibody is adalimumab. In another embodiment, the mannosylated N-glycan form is a Man 5 glycan. In one embodiment, said antibody comprises less than 5% Man 5 glycan. In another embodiment, said antibody comprises less than 4% Man 5 glycan. In yet another embodiment, said antibody comprises less than 3% Man 5 glycan. In yet another embodiment, said antibody comprises less than 2% Man 5 glycan. In yet another embodiment, said antibody comprises less than 1% Man 5 glycan. In one embodiment, said antibody comprises 4-5% Man 5 glycan. In another embodiment, said antibody comprises 3-4% Man 5 glycan. In another embodiment, said antibody comprises 2-3% Man 5 glycan. In another embodiment, said antibody comprises 1-2% Man 5 glycan. In another embodiment, said antibody comprises 0-1% Man 5 glycan. In a further embodiment, said antibody comprises less than 3% Man 5 glycan. In yet a further embodiment, said antibody comprises about 1.2% Man 5 glycan. In other embodiments, said antibody comprises less than about 4% Man 5 glycan, less than about 3.9% Man 5 glycan, less than about 3.8% Man 5 glycan, less than about 3.7% Man 5 glycan, less than about 3.6% Man 5 glycan, less than about 3.5% Man 5 glycan, less than about 3.4% Man 5 glycan, less than about 3.3% Man 5 glycan, less than about 3.2% Man 5 glycan, less than about 3.1% Man 5 glycan, less than about 3.0% Man 5 glycan, less than about 2.9% Man 5 glycan, less than about 2.8% Man 5 glycan, less than about 2.7% Man 5 glycan, less than about 2.6% Man 5 glycan, less than about 2.5% Man 5 glycan, less than about 2.4% Man 5 glycan, less than about 2.3% Man 5 glycan, less than about 2.2% Man 5 glycan, less than about 2.1% Man 5 glycan, less than about 2.0% Man 5 glycan, less than about 1.9% Man 5 glycan, less than about 1.8% Man 5 glycan, less than about 1.7% Man 5 glycan, less than about 1.6% Man 5 glycan, less than about 1.5% Man 5 glycan, less than about 1.4% Man 5 glycan, less than about 1.3% Man 5 glycan, less than about 1.2% Man 5 glycan, less than about 1.1% Man 5 glycan, less than about 1.0% Man 5 glycan, less than about 0.9% Man 5 glycan, less than about 0.8% Man 5 glycan, less than about 0.7% Man 5 glycan, less than about 0.6% Man 5 glycan, less than about 0.5% Man 5 glycan, less than about 0.4% Man 5 glycan, less than about 0.3% Man 5 glycan, less than about 0.2% Man 5 glycan, or less than about 0.1% Man 5 glycan.

In another aspect, the present invention provides compositions comprising an antibody comprising the heavy and light chain variable domains of adalimumab, wherein less than 5% of the total N-linked oligosaccharides present on said antibody are of a mannosylated N-glycan form. In one embodiment, the antibody is adalimumab. In another embodiment, the mannosylated N-glycan form is a Man 6 glycan. In a further embodiment, said antibody comprises 0.5-1% Man 6 glycan. In another embodiment, said antibody comprises 0.25-0.5% Man 6 glycan. In a further embodiment, said antibody comprises less than 0.25% Man 6 glycan. In other embodiments, said antibody comprises less than about 1% Man 6 glycan, less than about 0.9% Man 6 glycan, less than about 0.8% Man 6 glycan, less than about 0.7% Man 6 glycan, less than about 0.6% Man 6 glycan, less than about 0.5% Man 6 glycan, less than about 0.4% Man 6 glycan, less than about 0.3% Man 6 glycan, less than about 0.2% Man 6 glycan, or less than about 0.1% Man 6 glycan.

### BRIEF DESCRIPTION OF THE DRAWINGS

FIG. 1 depicts a graphical representation of the effects of dissolved oxygen on the viable cell density (VCD) for the adalimumab producing cell line 1 (media 2) at 35° C.

FIG. 2 depicts a graphical representation of the effects of dissolved oxygen on the viablity (%) for the adalimumab producing cell line 1 (media 2) at 35° C.

FIG. 3 depicts a graphical representation of the effects of dissolved oxygen on the harvest titer (g/L) for the adalimumab producing cell line 1 (media 2) at 35° C.

FIG. 4 depicts a graphical representation of the effects of dissolved oxygen on the percentage of oligomannose (Man5/6) for the adalimumab producing cell line 1 (media 2) at 35° C.

FIG. 5 depicts a graphical representation of the effects of dissolved oxygen on the viable cell density (VCD) for the adalimumab producing cell line 1 (media 2) at 33° C.

FIG. 6 depicts a graphical representation of the effects of dissolved oxygen on the viablity (%) for the adalimumab producing cell line 1 (media 2) at 33° C.

FIG. 7 depicts a graphical representation of the effects of dissolved oxygen on the harvest titer (g/L) for the adalimumab producing cell line 1 (media 2) at 33° C.

FIG. 8 depicts a graphical representation of the effects of dissolved oxygen on the percentage of oligomannose (Man5/6) for the adalimumab producing cell line 1 (media 2) at 33° C.

FIG. 9 depicts a graphical representation of the effects of dissolved oxygen on the viable cell density (VCD) for the adalimumab producing cell line 1 (media 1) at 35° C.

FIG. 10 depicts a graphical representation of the effects of dissolved oxygen on the viablity (%) for the adalimumab producing cell line 1 (media 1) at 35° C.

FIG. 11 depicts a graphical representation of the effects of dissolved oxygen on the harvest titer (g/L) for the adalimumab producing cell line 1 (media 1) at 35° C.

FIG. 12 depicts a graphical representation of the effects of dissolved oxygen on the percentage of oligomannose (Man5/6) for the adalimumab producing cell line 1 (media 1) at 35° C.

FIG. 13 depicts a graphical representation of the effects of dissolved oxygen on the viable cell density (VCD) for the adalimumab producing cell line 3 (media 1) at 33° C., 34° C., 35° C. and 36° C.

FIG. 14 depicts a graphical representation of the effects of dissolved oxygen on the viablity (%) for the adalimumab producing cell line 3 (media 1) at 33° C., 34° C., 35° C. and 36° C.

FIG. 15 depicts a graphical representation of the effects of dissolved oxygen on the harvest titer (g/L) for the adalimumab producing cell line 3 (media 1) at 33° C., 34° C., 35° C. and 36° C.

FIG. 16 depicts a graphical representation of the effects of dissolved oxygen on the percentage of oligomannose (Man5/6) for the adalimumab producing cell line 3 (media 1) at 33° C., 34° C., 35° C. and 36° C.

FIG. 17 depicts a graphical representation of the effects of dissolved oxygen on the viable cell density (VCD) for the mAB2 producing cell line (media 1) at 35° C.

FIG. 18 depicts a graphical representation of the effects of dissolved oxygen on the viablity (%) for the mAB2 producing cell line (media 1) at 35° C.

FIG. 19 depicts a graphical representation of the effects of dissolved oxygen on the harvest titer (g/L) for the mAB2 producing cell line (media 1) at 35° C.

FIG. 20 depicts a graphical representation of the effects of dissolved oxygen on the percentage of oligomannose (Man5/6) for the mAB2 producing cell line (media 1) at 35° C.

### DETAILED DESCRIPTION OF THE INVENTION

The present invention provides methods and compositions for modulating the glycosylation profile of a protein such as a therapeutic protein (e.g., an antibody such as adalimumab).

The present invention is based on the identification and optimization of upstream process technologies, e.g., recombinant cell culture conditions, for protein production, e.g., production of antibodies or antigen-binding portions thereof, resulting in the production of protein compositions with modulated glycosylation profiles (e.g., increased or decreased mannosylation, modulated galactosylation and/or modulated levels of agalactosyl N-glycans).

### I. Definitions

In order that the present invention may be more readily understood, certain terms are first defined.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms, for example, those characterized by “a” or “an”, shall include pluralities, e.g., one or more impurities. In this application, the use of “or” means “and/or”, unless stated otherwise. Furthermore, the use of the term “including,” as well as other forms of the term, such as “includes” and “included”, is not limiting. Also, terms such as “element” or “component” encompass both elements and components comprising one unit and elements and components that comprise more than one unit unless specifically stated otherwise.

Most naturally occurring peptides (or proteins) comprise carbohydrate or saccharide moieties attached to the peptide via specific linkages to a select number of amino acids along the length of the primary peptide chain. Thus, many naturally occurring peptides are termed “glycopeptides” or “glycoproteins” or are referred to as “glycosylated” proteins or peptides.

The term “glycoform” refers an isoform of a protein, e.g., an antibody, that differs only with respect to the number and/or type of attached glycan(s). Glycoproteins often consist of a number of different glycoforms.

The predominant sugars found on glycoproteins are glucose, galactose, mannose, fucose, N-acetylgalactosamine (“GalNAc”), N-acetylglucosamine (“GlcNAc”) and sialic acid (e.g., N-acetylneuraminic acid (“NANA” or “NeuAc”, where “Neu” is neuraminic acid) and “Ac” refers to “acetyl”). The processing of the sugar groups occurs co-translationally in the lumen of the ER and continues in the Golgi apparatus for N-linked glycoproteins.

The oligosaccharide structure attached to the peptide chain is known as a “glycan” molecule. The glycan structures found in naturally occurring glycopeptides are typically divided into two classes, “N-linked glycans” or “N-linked oligosaccharides” and “O-linked glycans” or “O-linked oligosaccharides”.

Peptides expressed in eukaryotic cells typically comprise N-glycans. “N-glycans” are N-glycosylated at an amide nitrogen of an asparagine or an arginine residue in a protein via an N-acetylglucosamine residue. These “N-linked glycosylation sites” occur in the peptide primary structure containing, for example, the amino acid sequence asparagine-X-serine/threonine, where X is any amino acid residue except proline and aspartic acid.

Techniques for the determination of glycan primary structure are well known in the art and are described in detail, for example, in Montreuil, “Structure and Biosynthesis of Glycopeptides” In Polysaccharides in Medicinal Applications, pp. 273-327, 1996, Eds. Severian Damitriu, Marcel Dekker, NY. It is therefore a routine matter for one of ordinary skill in the art to isolate a population of peptides produced by a cell and determine the structure(s) of the glycans attached thereto. For example, efficient methods are available for (i) the splitting of glycosidic bonds either by chemical cleavage such as hydrolysis, acetolysis, hydrazinolysis, or by nitrous deamination; (ii) complete methylation followed by hydrolysis or methanolysis and by gas-liquid chromatography and mass spectroscopy of the partially methylated monosaccharides; and (iii) the definition of anomeric linkages between monosaccharides using exoglycosidases, which also provide insight into the primary glycan structure by sequential degradation. Flouresecent labeling and subsequent high performance liquid chromatography (HPLC), e.g., normal phase HPLC (NP-HPLC), mass spectroscopy and nuclear magnetic resonance (NMR) spectrometry, e.g., high field NMR, may also be used to determine glycan primary structure.

Kits and equipment for carbohydrate analysis are also commercially available. Fluorophore Assisted Carbohydrate Electrophoresis (FACE) is available from Glyko, Inc. (Novato, Calif.). In FACE analysis, glycoconjugates are released from the peptide with either Endo H or N-glycanase (PNGase F) for N-linked glycans, or hydrazine for Ser/Thr linked glycans. The glycan is then labeled at the reducing end with a fluorophore in a non-structure discriminating manner. The fluorophore labeled glycans are then separated in polyacrylamide gels based on the charge/mass ratio of the saccharide as well as the hydrodynamic volume. Images are taken of the gel under UV light and the composition of the glycans is determined by the migration distance as compared with the standards. Oligosaccharides can be sequenced in this manner by analyzing migration shifts due to the sequential removal of saccharides by exoglycosidase digestion.

All N-linked oligosaccharides have a common “pentasaccharide core” of Man₃GlcNAc₂. (“Man” refers to mannose; “Glc” refers to glucose; “NAc” refers to N-acetyl; and “GlcNAc” refers to N-acetylglucosamine). The pentasaccharide core is also referred to as the “trimannose core” or the “paucimannose core”.

N-glycans differ with respect to the presence of, and/or in the number of branches (also called “antennae”) comprising peripheral sugars such as N-acetylglucosamine, galactose, N-acetylgalactosamine, N-acetylneuraminic acid, fucose and sialic acid that are added to the Man₃GlcNAc₂ core structure. Optionally, this structure may also contain a core fucose molecule and/or a xylose molecule. For a review of standard glycobiology nomenclature see, Essentials of Glycobiology Varki et al. eds., 1999, CSHL Press, the contents of which are incorporated herein by reference.

N-glycans are classified according to their branched constituents (e.g., oligomannose-type, complex, or hybrid). An “oligomannose-type” or “high mannose-type” N-glycan has five or more mannose residues.

A “complex-type” N-glycan typically has at least one GlcNAc attached to the 1,3 mannose arm and at least one GlcNAc attached to the 1,6 mannose arm of a pentasaccharide core. Complex-type N-glycans may also have galactose (“Gal”) or N-acetylgalactosamine residues that are optionally modified with sialic acid or derivatives, e.g., N-acetyl neuraminic acid. Complex-type N-glycans may also have intrachain substitutions comprising “bisecting” GlcNAc, and core fucose (“Fuc”). Complex N-glycans may also have multiple antennae on the pentasaccharide core and are, therefore, also referred to as “multiple antennary-type glycans.”

A “hybrid-type” N-glycan comprises at least one GlcNAc on the terminal of the 1,3 mannose arm of the pentasaccharide core and zero or more mannoses on the 1,6 mannose arm of the trimannose core.

The oligomannose-type structures that may be present within the compositions of the invention and/or may be used in the methods of the invention are referred to herein as “M5”, “Man 5” or “Man 5 glycan”; “M6”, “Man 6” or “Man 6 glycan”; “M7”, “Man 7” or “Man 7 glycan”; “M8”, “Man 8” or “Man 8 glycan”; and “M9”, “Man 9” or “Man 9 glycan.”

In one embodiment, an M5 oligomannose-type structure has the structure (I):

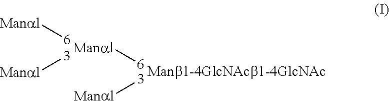

In one embodiment, an M6 oligomannose-type structure has the structure (II):

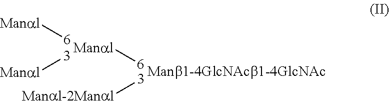

In one embodiment, an M7 oligomannose-type structure has the structure (III):

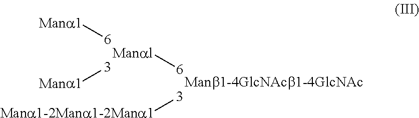

In another embodiment, an M7 oligomannose-type structure has the structure (IV):

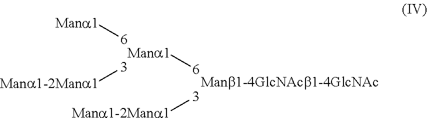

In another embodiment, an M7 oligomannose-type structure has the structure (V):

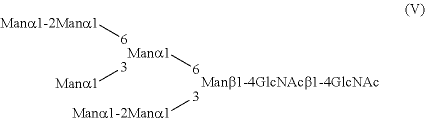

In one embodiment, an M8 oligomannose-type structure has the structure (VI):

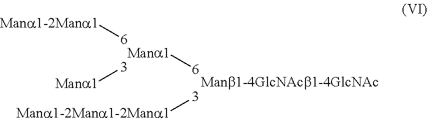

In another embodiment, an M8 oligomannose-type structure has the structure (VII):

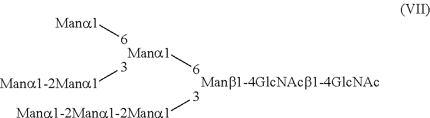

In another embodiment, an M8 oligomannose-type structure has the structure (VIII):

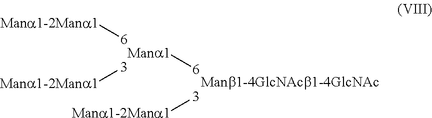

In one embodiment, an M9 oligomannose-type structure has the structure (IX):

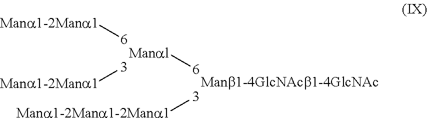

In one embodiment, the oligomannose-type structures that may be present within the compositions of the invention and/or may be used in the methods of the invention are independently selected from the group consisting of Man 5 glycan, Man 6 glycan, Man 7 glycan, Man 8 glycan, and/or Man 9 glycan.

In one embodiment, a multiple antennary-type structure that may be present within the compositions of the invention and/or may be used in the methods of the invention is a “bianntennary oligosaccharide-type structure”. A “bianntennary oligosaccharide-type structure” is an N-linked glycan having two branches or arms, and a core fucose with zero, one or two galactose additions on the arms. In one embodiment, a “bianntennary oligosaccharide-type structure” that may be present within the compositions of the invention and/or may be used in the methods of the invention is bisected. In one embodiment, a “bianntennary oligosaccharide-type structure” that may be present within the compositions of the invention and/or may be used in the methods of the invention is a “fucosylated bianntennary oligosaccharide-type structure”, e.g., comprises a core-substituted with fucose.

In one embodiment, a “fucosylated bianntennary oligosaccharide-type structure” that may be present within the compositions of the invention and/or may be used in the methods of the invention is an “asialo, fucosylated bianntennary oligosaccharide-type structure”, also referred to as an “asialo, bigalactosylated biantennary, core-substituted with fucose”, referred to herein as“NA2F” or “G2F.”

In another embodiment, a “fucosylated bianntennary oligosaccharide-type structure” that may be present within the compositions of the invention and/or may be used in the methods of the invention is a asialo, agalacto, fucosylated bianntennary oligosaccharide-type structure, also referred to as an asialo, agalacto-, biantennary, core-substituted with fucose, referred to herein as “NGA2F” or “G0F.”

In another embodiment, a “fucosylated bianntennary oligosaccharide-type structure” that may be present within the compositions of the invention and/or may be used in the methods of the invention is a asialo, fucosylated bianntennary oligosaccharide-type structure, also referred to as asialo, monogalactosylated biantennary, core-substituted with fucose, referred to herein as “NA1F” or “G1F.”

In another embodiment, a “fucosylated bianntennary oligosaccharide-type structure” that may be present within the compositions of the invention and/or may be used in the methods of the invention is a asialo, agalacto, fucosylated biantennary, minus a bisecting N-acetylglucosamine oligosaccharide-type structure, also referred to as asialo, agalacto-, biantennary, core-substituted with fucose minus a bisecting N-acetylglucosamine, referred to herein as “NGA2F-GlcNAc” or “G0F-GlcNAc.”

In yet another embodiment, a “fucosylated bianntennary oligosaccharide-type structure” that may be present within the compositions of the invention and/or may be used in the methods of the invention is a asialo, monogalacto, fucosylated biantennary, minus a bisecting N-acetylglucosamine oligosaccharide-type structure, also referred to as asialo, monogalactosylated biantennary, core-substituted with fucose minus a bisecting N-acetylglucosamine, referred to herein as “NA1F-GlcNAc” or “G1F-GlcNAc.”

In one embodiment, an NA2F, or a G2F, fucosylated biantennary oligosaccharide-type structure has the structure (X):

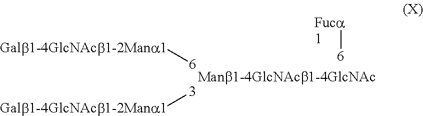

In one embodiment, an NGA2F, or a G0F, fucosylated biantennary oligosaccharide-type structure has the structure (XI):

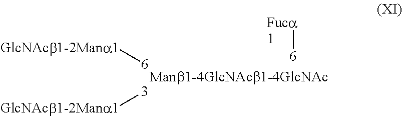

In one embodiment, an NA1F, or a G1F, fucosylated biantennary oligosaccharide-type structure has the structure (XII):

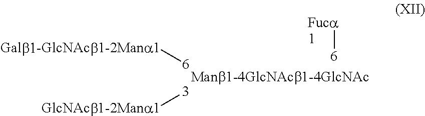

In another embodiment, an NA1F, or a G1F, fucosylated biantennary oligosaccharide-type structure has the structure (XIII)

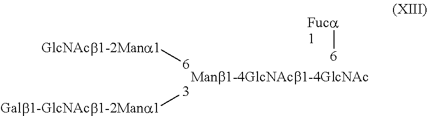

In one embodiment, an NGA2F-GlcNAc, or a G0F-GlcNAc, and NA1F-G1cNAc fucosylated biantennary oligosaccharide-type structure has the structure (XIV):

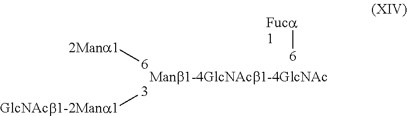

In one embodiment, an NA1F-G1cNAc, or a G1F-GlcNAc, fucosylated biantennary oligosaccharide-type structure has the structure (XV):

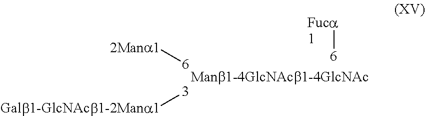

In one embodiment, the fucosylated biantennary oligosaccharide-type structure is independently selected from the group consisting of NGA2F, NA1F, NA2F, NGA2F-GlcNAc, and NA1F-GlcNAc.

In another embodiment, a multiple antennary-type structure that may be present within the compositions of the invention and/or may be used in the methods of the invention is a “bianntennary oligosaccharide-type structure” N-linked glycan having two branches or arms, with zero, one or two galactose additions on the arms. In one embodiment, a “bianntennary oligosaccharide-type structure” that may be present within the compositions of the invention and/or may be used in the methods of the invention is a “asialo, agalacto bianntennary oligosaccharide-type structure” referred herein as “NGA2” or “G0.” In one embodiment, an NGA2, or G0, biantennary oligosaccharide-type structure has the structure (XVI):

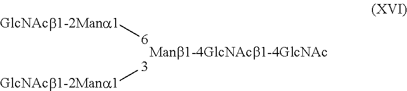

In one embodiment, a “bianntennary oligosaccharide-type structure” that may be present within the compositions of the invention and/or may be used in the methods of the invention is a “asialo, agalacto bianntennary oligosaccharide-type structure” referred herein as NGA1, or G0-GlcNAc. In one embodiment, an NGA1, or a G0-GlcNAc, biantennary oligosaccharide-type structure has the structure (XVII):

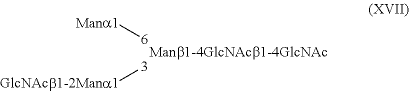

As used herein, “high mannose” includes the amount or level of mannosylation, or mannosylated N-glycans, in the recombinant protein, including, for example, Man 9, Man 8, Man 7, Man 6 and Man 5.

As used herein, a “modulated glycosylation profile” includes a glycosylation profile of a composition comprising a recombinant protein (e.g., an antibody, such as adalimumab) which is modulated as compared to the glycosylation profile of a composition comprising that same recombinant protein produced by culturing a host cell expressing that recombinant protein in cell culture media which is not supplemented with dissolved oxygen. The modulated glycosylation profile may include an overall modulation (e.g., an increase or decrease) in the level of high mannose N-glycans, a modulation (e.g., an increase or decrease) in the level of galactosylated N-glycans and/or modulation (e.g., an increase or decrease) in the levels of agalactosyl N-glycans in the recombinant protein. For example, the overall amount or level of mannosylated N-glycans in the recombinant protein may be increased or decreased by about 0.1%, 1%, 1.2%, 1.5%, 2%, 2.2%, 2.5%, 2.55%, 3%, 3.2%, 3.5%, 4%, 4.2%, 4.5%, 5%, 10%, 15%, 20%, 25% or 30%. Ranges within one or more of the preceding values, e.g., about 0.1-5%, 0.1-10%, 0.1-15%, 0.1-20%, 0.1-25%, 0.1-30%, 1-5%, 1-10%, 1-15%, 1-20%, 1-25%, 1-30%, 2-5%, 2-10%, 2-15%, 2-20%, 2-25%, 2-30%, 3-5%, 3-10%, 3-15%, 3-20%, 3-25%, 3-30%, 4-5%, 4-10%, 4-15%, 4-20%, 4-25% or 4-30% are contemplated by the invention.

In another example, the overall amount or level of mannosylated N-glycans comprises an increase in the amount or level of a high mannose N-glycan oligosaccharide. A high-mannose N-glycan has more than one mannose linked to the non-reducing terminal of the core structure. For example, the high mannose N-glycan oligosaccharide is selected from the group consisting of Man 5 glycan, Man 6 glycan, Man 7 glycan and Man 8 glycan. In one embodiment, the amount or level of at least one of Man 5 glycan, Man 6 glycan, Man 7 glycan and/or Man 8 glycan is increased by about 0.1%, 1%, 1.2%, 1.5%, 2%, 2.2%, 2.5%, 3%, 3.2%, 3.5%, 4%, 4.2%, 4.5%, 5%, 10%, 15%, 20%, 25%, or 30%. Ranges within one or more of the preceding values, e.g., about 0.1-5%, 0.1-10%, 0.1-15%, 0.1-20%, 0.1-25%, 0.1-30%, 1-5%, 1-10%, 1-15%, 1-20%, 1-25%, 1-30%, 2-5%, 2-10%, 2-15%, 2-20%, 2-25%, 2-30%, 3-5%, 3-10%, 3-15%, 3-20%, 3-25%, 3-30%, 4-5%, 4-10%, 4-15%, 4-20%, 4-25% or 4-30% are contemplated by the invention.

In another example, the overall amount or level of mannosylated N-glycans comprises a decrease in the amount or level of a high mannose N-glycan oligosaccharide. A high-mannose N-glycan has more than one mannose linked to the non-reducing terminal of the core structure, as noted above. For example, the high mannose N-glycan oligosaccharide is selected from the group consisting of Man 5 glycan, Man 6 glycan, Man 7 glycan and Man 8 glycan. In one embodiment, the amount or level of at least one of Man 5 glycan, Man 6 glycan, Man 7 glycan and/or Man 8 glycan is decreased by about 0.1%, 1%, 1.2%, 1.5%, 2%, 2.2%, 2.5%, 2.55%, 3%, 3.2%, 3.5%, 4%, 4.2%, 4.5%, 5%, 10%, 15%, 20%, 25%, or 30%. Ranges within one or more of the preceding values, e.g., about 0.1-3%, 0.1-5%, 0.1-10%, 0.1-15%, 0.1-20%, 0.1-25%, 0.1-30%, 1-5%, 1-10%, 1-15%, 1-20%, 1-25%, 1-30%, 2-5%, 2-10%, 2-15%, 2-20%, 2-25%, 2-30%, 3-5%, 3-10%, 3-15%, 3-20%, 3-25%, 3-30%, 4-5%, 4-10%, 4-15%, 4-20%, 4-25% or 4-30% are contemplated by the invention.

In another example, an overall increase in the amount or level of galactosylation in the recombinant protein, or galactosylated N-glycans, in the recombinant protein, resulting from modulation of any one of the galactosylated glycan species such as G1F, G1F-GlcNAc and/or G2F is increased by about 0.1%, 1%, 1.2%, 1.5%, 2%, 2.2%, 2.5%, 3%, 3.2%, 3.5%, 4%, 4.2%, 4.5%, 5%, 10%, 15%, 20%, 25%, 30%, 35%, 40%, 45% or 50%, 55%, 60%. Ranges within one or more of the preceding values, e.g., about 0.1-5%, 0.1-10%, 0.1-15%, 0.1-20%, 0.1-25%, 0.1-30%, 0.1-35%, 0.1-40%, 0.1-45%, 0.1-50%, 0.1-55%, 0.1-60%, 1-5%, 1-10%, 1-15%, 1-20%, 1-25%, 1-30%, 1-35%, 1-40%, 1-45%, 1-50%, 1-55%, 1-60%, 2-5%, 2-10%, 2-15%, 2-20%, 2-25%, 2-30%, 2-35%, 2-40%, 2-45%, 2-50%, 2-55%, 2-60%, 3-5%, 3-10%, 3-15%, 3-20%, 3-25%, 3-30%, 3-35%, 3-40%, 3-45%, 3-50%, 3-55%, 3-60%, 4-5%, 4-10%, 4-15%, 4-20%, 4-25%, 4-30%, 4-35%, 4-40%, 4-45%, 4-50%, 4-55%, 4-60%, 5-10%, 5-15%, 5-20%, 5-25%, 5-30%, 5-35%, 5-40%, 5-45%, 5-50%, 5-55% or 5-60% are contemplated by the invention.

In another example, an overall decrease in the amount or level of agalactosyl N-glycans in the recombinant protein resulting from modulation of any one of the agalactosyl N-glycan species such as G0, G0-GlcNAc, G0F and/or G0F-GlcNAc is decreased by about 0.1%, 1%, 1.2%, 1.5%, 2%, 2.2%, 2.5%, 3%, 3.2%, 3.5%, 4%, 4.2%, 4.5%, 5%, 10%, 15%, 20%, 25%, 30%, 35%, 40% or 45%. Ranges within one or more of the preceding values, e.g., about 0.1-5%, 0.1-10%, 0.1-15%, 0.1-20%, 0.1-25%, 0.1-30%, 0.1-35%, 0.1-40%, 0.1-45%, 1-5%, 1-10%, 1-15%, 1-20%, 1-25%, 1-30%, 2-5%, 2-10%, 2-15%, 2-20%, 2-25%, 2-30%, 2-35%, 2-40%, 2-45%, 3-5%, 3-10%, 3-15%, 3-20%, 3-25%, 3-30%, 3-35%, 3-40%, 3-45%, 4-5%, 4-10%, 4-15%, 4-20%, 4-25%, 4-30%, 4-35%, 4-40%, 4-45%, 5-10%, 5-15%, 5-20%, 5-25%, 5-30%, 5-35%, 5-40% or 5-45% are contemplated by the invention.

The term “level” with respect to protein such as an antibody, or antigen-binding fragment thereof, which is glycosylated at an N-linked glycosylation site on the Fc region in a composition refers to the relation of one glycoform in the composition to the whole of the glycoform levels in the composition and is expressed as a percentage of the whole, e.g., 0-100%. The level in a composition may be an absolute amount as measured in molecules, moles, or weight percent.

Compositions comprising varying levels of glycoforms of a protein such as a human antibody, or antigen-binding fragment thereof, are useful in that by varying the glycoform compositions a desired characteristics, e.g., rate of serum clearance or ADCC activity, may be achieved.

The methods of the invention can be used to produce compositions of any protein, such as a therapeutic protein, e.g., an antibody, an antigen-binding portion thereof, a DVD-Ig, a TVD-Ig, a RAB or a half-body.

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antigen-binding portion” of an antibody (or “antibody portion”) includes fragments of an antibody that retain the ability to specifically bind to an antigen (e.g., in the case of Adalimumab, hTNFα). It has been shown that the antigen-binding function of an antibody can be performed by fragments of a full-length antibody. Examples of binding fragments encompassed within the term “antigen-binding portion” of an antibody include (i) a Fab fragment, a monovalent fragment comprising the VL, VH, CL and CHI domains; (ii) a F(ab′)2 fragment, a bivalent fragment comprising two Fab fragments linked by a disulfide bridge at the hinge region; (iii) a Fd fragment comprising the VH and CHI domains; (iv) a Fv fragment comprising the VL and VH domains of a single arm of an antibody, (v) a dAb fragment (Ward et al., (1989) Nature 341:544-546, the entire teaching of which is incorporated herein by reference), which comprises a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883, the entire teachings of which are incorporated herein by reference). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123, the entire teachings of which are incorporated herein by reference). Still further, an antibody or antigen-binding portion thereof may be part of a larger immunoadhesion molecule, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101, the entire teaching of which is incorporated herein by reference) and use of a cysteine residue, a marker peptide and a C-terminal polyhistidine tag to make bivalent and biotinylated scFv molecules (Kipriyanov, S. M., et al. (1994) Mol Immunol. 31:1047-1058, the entire teaching of which is incorporated herein by reference). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. In one aspect, the antigen binding fragments are complete domains or pairs of complete domains.

The term “human antibody” includes antibodies having variable and constant regions corresponding to human germline immunoglobulin sequences as described by Kabat et al. (See Kabat, et al. (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), e.g., in the CDRs and in particular CDR3. The mutations can be introduced using the “selective mutagenesis approach.” The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immunoglobulin sequence. In other embodiments, up to ten, up to five, up to three or up to two positions are replaced. In one embodiment, these replacements are within the CDR regions. However, the term “human antibody”, as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see, e.g., Taylor, L. D., et al. (1992) Nucl. Acids Res. 20:6287-6295, the entire teaching of which is incorporated herein by reference) or antibodies prepared, expressed, created or isolated by any other means that involves splicing of human immunoglobulin gene sequences to other DNA sequences. Such recombinant human antibodies have variable and constant regions derived from human germline immunoglobulin sequences (see, Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or back-mutation or both.

An “isolated antibody” includes an antibody that is substantially free of other antibodies having different antigenic specificities (e.g., an isolated antibody that specifically binds hTNFα is substantially free of antibodies that specifically bind antigens other than hTNFα). An isolated antibody that specifically binds hTNFα may bind TNFα molecules from other species. Moreover, an isolated antibody may be substantially free of other cellular material and/or chemicals. A suitable anti-TNFα antibody is adalimumab.

As used herein, the term “adalimumab,” also known by its trade name HUMIRA® (AbbVie) refers to a human IgG₁ antibody that binds human tumor necrosis factor α (TNFα). In general, the heavy chain constant domain 2 (CH2) of the adalimumab IgG-Fc region is glycosylated through covalent attachment of oligosaccharide at asparagine 297 (Asn-297). The light chain variable region of adalimumab is provided herein as SEQ ID NO:1, and the heavy chain variable region of adalimumab is provided herein as SEQ ID NO:2. Adalimumab comprises a light chain variable region comprising a CDR1 of SEQ ID NO:7, a CDR2 of SEQ ID NO:5, and a CDR3 of SEQ ID NO:3. Adalimumab comprises a heavy chain variable region comprising a CDR1 of SEQ ID NO:8, a CDR2 of SEQ ID NO:6 and CDR3 of SEQ ID NO:4. The nucleic acid sequence of the light chain variable region is set forth in SEQ ID NO:9. The nucleic acid sequence of the heavy chain variable region is set forth in SEQ ID NO:10. The full length amino acid sequence of the light chain is set forth as SEQ ID NO:11 and the full length amino acid sequence of the heavy chain is set forth as SEQ ID NO:12. Adalimumab is described in U.S. Pat. Nos. 6,090,382; 6,258,562; 6,509,015; 7,223,394; 7,541,031; 7,588,761; 7,863,426; 7,919,264; 8,197,813; 8,206,714; 8,216,583; 8,420,081; 8,092,998; 8,093,045; 8,187,836; 8,372,400; 8,034,906; 8,436,149; 8,231,876; 8,414,894; 8,372,401, the entire contents of each which are expressly incorporated herein by reference in their entireties. Adalimumab is also described in the “Highlights of Prescribing Information” for HUMIRA® (adalimumab) Injection (Revised January 2008) the contents of which are hereby incorporated herein by reference.

As used herein, a heavy chain antigen binding domain (referred to herein as VD or VDH) is intended to include a heavy chain variable domain, a dual heavy chain variable domain, a triple heavy chain variable domain, a light chain variable domain, a dual light chain variable domain, a triple light chain variable domain, a heavy chain variable domain in combination with a light chain variable domain, two heavy chain variable domains in combination with a light chain variable domain, a heavy chain variable domain in combination with two light chain variable domains, a domain antibody, a camelid antibody, a scFv, a receptor, and a scaffold antigen binding protein. It is understood that the heavy chain antigen binding domain may or may not bind an antigen independently of a paired light chain, dual light chain, or triple light chain, as appropriate, present on a second polypeptide of the binding proteins of the invention. For example, a domain antibody, a scFv, or a receptor would be expected to bind a target independent of any amino acid sequences on a second polypeptide claim. As the binding proteins of the invention form functional antigen binding sites, if the heavy chain antigen binding domain cannot specifically bind a target antigen independently (i.e., does not alone provide a functional antibody binding site), a second polypeptide should be present to provide a complementary light chain variable domain to provide a functional antibody binding site.

As used herein, a light chain antigen binding domain (referred to herein as VD or VDL) is intended to include a light chain variable domain, a dual light chain variable domain, a triple light chain variable domain, a heavy chain variable domain, a dual heavy chain variable domain, a triple heavy chain variable domain, a heavy chain variable domain in combination with a light chain variable domain, two heavy chain variable domains in combination with a light chain variable domain, a heavy chain variable domain in combination with two light chain variable domains, a camelid antibody, a domain antibody, a camelid antibody, a scFv, a receptor, and a scaffold antigen binding protein. It is understood that the light chain antigen binding domain may or may not bind an antigen independently of a paired heavy chain, dual heavy chain, or triple heavy chain, as appropriate, present on another polypeptide of the binding proteins of the invention. For example, a domain antibody, a scFv, or a receptor would be expected to bind a target independent of any amino acid sequences on a second polypeptide claim.

As used herein, “VD” alone can be understood to be either a heavy chain antigen binding domain or a light chain antigen binding domain unless otherwise clear from context.

As used herein, “Dual Variable Domain Immunoglobulin” or “DVD-Ig™” and the like are provided in US Patent Publications 20100260668 and 20090304693 both of which are incorporated herein by reference. DVDs may be monospecific, i.e., bind one antigen, or multispecific, i.e. bind two or more antigens. A DVD-Ig™ comprises a paired heavy chain DVD polypeptide and a light chain DVD polypeptide with each paired heavy and light chain providing two antigen binding sites. Each binding site includes a total of 6 CDRs involved in antigen binding per antigen binding site. A DVD-Ig™ is typically has two arms bound to each other at least in part by dimerization of the CH3 domains, with each arm of the DVD being bispecific, providing an immunoglobulin with four binding sites.

A TVD-Ig is described in PCT Publication No. WO 2012/088290, the entire contents of which are incorporated herein by reference. A half-body is described in PCT Publication No. WO 2012/088302, the entire contents of which are incorporated herein by reference.

As used herein, the term “upstream process technology,” in the context of protein, e.g., antibody, preparation, refers to activities involving the production and collection of proteins (e.g. antibodies or DVD-Igs) from cells (e.g., during cell culture of a protein with a modulated glycosylation profile). As used herein, the term “cell culture” refers to methods and techniques employed to generate and maintain a population of host cells capable of producing a recombinant protein with a modulated glycosylation profile, as well as the methods and techniques for optimizing the production and collection of the protein with a modulated glycosylation profile. For example, once an expression vector has been incorporated into an appropriate host, the host can be maintained under conditions suitable for high level expression of the relevant nucleotide coding sequences, and the collection and purification of the desired recombinant protein.

When using the cell culture techniques of the instant invention, the protein with a modulated glycosylation profile can be produced intracellularly, in the periplasmic space, or directly secreted into the medium. In embodiments where the protein with a modulated glycosylation profile is produced intracellularly, the particulate debris, either host cells or lysed cells (e.g., resulting from homogenization), can be removed by a variety of means, including but not limited to, by centrifugation or ultrafiltration. Where the protein with a modulated glycosylation profile is secreted into the medium, supernatants from such expression systems can be first concentrated using a commercially available protein concentration filter, e.g., an Amicon™ or Millipore Pellicon™ ultrafiltration unit.

As used herein, the term “downstream process technology” refers to one or more techniques used after the upstream process technologies to purify the protein, e.g., antibody, antigen-binding portion thereof, or DVD-Ig, of interest. For example, downstream process technology includes purification of the protein product, using, for example, affinity chromatography, including Protein A affinity chromatography, ion exchange chromatography, such as anion or cation exchange chromatography, hydrophobic interaction chromatography, displacement chromatography, multi-mode chromatography, continuous and recycle chromatography, viral filtration, depth filtration, ultrafiltration, diafiltration and centrifugation.

As used herein a “recombinant expression vector” can be any suitable recombinant expression vector, and can be used to transform or transfect any suitable host. For example, one of ordinary skill in the art would appreciate that transformation or transfection is a process by which exogenous nucleic acid such as DNA is introduced into a cell wherein the transformation or transfection process involves contacting the cell with the exogenous nucleic acid such as the recombinant expression vector as described herein. Non-limiting examples of such expression vectors are the pUC series of vectors (Fermentas Life Sciences), the pBluescript series of vectors (Stratagene, LaJolla, Calif.), the pET series of vectors (Novagen, Madison, Wis.), the pGEX series of vectors (Pharmacia Biotech, Uppsala, Sweden), and the pEX series vectors (Clontech, Palo Alto, Calif.).

The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells selected from any of the Kingdoms of life. In another embodiment, eukaryotic cells include protist, fungal, plant and animal cells. In another embodiment, host cells include, but are not limited to, the prokaryotic cell line E. coli; mammalian cell lines CHO, HEK 293, COS, NS0, SP2 and PER.C6; the insect cell line Sf9; and the fungal cell Saccharomyces cerevisiae.

In certain embodiments, the host cells used in the methods of the present invention are prokaryote, yeast, or higher eukaryote cells. Suitable prokaryotes for this purpose include eubacteria, such as Gram-negative or Gram-positive organisms, e.g., Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One suitable E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In certain embodiments, the host cells are eukaryotic microbes such as filamentous fungi or yeast. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

In certain embodiments the host cells are derived from multicellular organisms. In particular embodiments, the cells are invertebrate cells from plant and insect cells. Non-limiting examples include cells derived from Spodoptera frugiperda (caterpillar), Aedes aegypti (mosquito), Aedes albopictus (mosquito), Drosophila melanogaster (fruitfly), Bombyx mori, cotton, corn, potato, soybean, petunia, tomato, and tobacco can also be utilized.

As used herein, the term “recombinant protein” refers to a protein produced as the result of the transcription and translation of a gene carried on a recombinant expression vector that has been introduced into a host cell. In certain embodiments the recombinant protein is an antibody, preferably a chimeric, humanized, or fully human antibody. In certain embodiments the recombinant protein is an antibody of an isotype selected from group consisting of: IgG (e.g., IgG1, IgG2, IgG3, IgG4), IgM, IgA1, IgA2, IgD, or IgE. In certain embodiments the antibody molecule is a full-length antibody (e.g., an IgG1 or IgG4 immunoglobulin) or alternatively the antibody can be a fragment (e.g., an Fc fragment or a Fab fragment). In some embodiments, the recombinant protein is a DVD-Ig, a TVD-Ig, a RAB or a half-body.

The term “about”, as used herein, is intended to refer to ranges of approximately 0.1-2.0% greater than or less than the referenced value. In certain circumstances, one of skill in the art will recognize that, due to the nature of the referenced value, the term “about” can mean more or less than a 0.1-2.0% deviation from that value.

The term “control”, as used herein, is intended to refer to a composition comprising a protein produced by culturing a host cell expressing a protein in cell culture media which is not supplemented with dissolved oxygen. For example, a control may include a composition comprising a protein (e.g., an antibody) produced using the same host cell line and the same recombinant expression vector under the same cell culture conditions, including the same culture media, same culture vessel, same culture mode, same culture temperature and same pH, but without dissolved oxygen supplementation. For example, if antibody X is the antibody whose glycosylation profile is modulated using the methods of the invention, the control would be a composition comprising antibody X produced using the same host cell line and the same recombinant expression vector under the same cell culture conditions, including the same culture media, same culture vessel, same culture mode, same culture temperature and same pH, but without dissolved oxygen supplementation.

The terms “dissolved oxygen” and “DO” are used interchangeably herein to refer to the amount (volume) of oxygen that is dissolved in a particular volume of medium. Oxygen can be dissolved in a particular medium using methods known in the art (e.g., through agitation and aeration of the medium by stirring or shaking). For example, 10% DO is intended to refer to a medium comprising 10% by volume of dissolved oxygen; 20% DO is intended to refer to a medium comprising 20% by volume of dissolved oxygen; 30% DO is intended to refer to a medium comprising 30% by volume of dissolved oxygen; 40% DO is intended to refer to a medium comprising 40% by volume of dissolved oxygen; 50% DO is intended to refer to a medium comprising 50% by volume of dissolved oxygen; 60% DO is intended to refer to a medium comprising 60% by volume of dissolved oxygen; 70% DO is intended to refer to a medium comprising 70% by volume of dissolved oxygen; and 80% DO is intended to refer to a medium comprising 80% by volume of dissolved oxygen.

### II. Modulation of Recombinant Protein Glycosylation Using Dissolved Oxygen Glycosylation

The pattern of glycoforms that arise in recombinant proteins, including monoclonal antibodies, can be affected by culture conditions during production (Nam et al. (2008) Biotechnol. Bioeng. 100(6): 1178-92). Consistency in the quality of the glycoproteins is important as glycosylation may impact protein solubility, activity, and circulatory half-life. (Gawlitzek et al. (1995) Biotechnol. Bioeng. 46:536-544; and Hayter et al. (1992) Biotechnol. Bioeng. 39:327-335).

Post-translational modification of nascent recombinant proteins includes enzymatic glycosylation. The resulting proteins, bearing covalently linked oligosaccharide side chains, are known as glycosylated proteins or glycoproteins. Antibodies are glycoproteins with one or more carbohydrate residues in the Fc domain, as well as the variable domain. Carbohydrate residues in the Fc domain have an important effect on the effector function of the Fc domain, with minimal effect on antigen binding or half-life of the antibody (Jefferis, R. Biotechnol. Prog. (2005) 21:11-16). In contrast, glycosylation of the variable domain may have an effect on the antigen binding activity of the antibody. Glycosylation in the variable domain may also have a negative effect on antibody binding affinity, likely due to steric hindrance (Co, M. S. et al., (1993) Mol. Immunol. 30:1361-1367), or result in increased affinity for the antigen (Wallick, S.C. et al., (1988) Exp. Med. 168:1099-1109; Wright, A. et al., (1991) EMBO J. 10:2717 2723).

Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the proteins produced using the methods of the present invention may include, but are not limited to, glucose, galactose, mannose, fucose, n-acetylglucosamine, NGA2F-GlcNAc (also referred to as G0F-GlcNAc), NGA2F (also referred to as G0F), NA1F-GlcNAc (also referred to as G1F-GlcNAc), NA1F (also referred to as G1F), NA2F (also referred to as G2F), NGA1 (also referred to as G0-GlcNAc), NGA2 (also referred to as G0) and sialic acid.

In one aspect of the present invention, the glycosylation of a protein, e.g., an antibody, such as adalimumab, an antigen-binding portion thereof, or a DVD-Ig, is modulated. Glycosylation can be modulated to, for example, increase the affinity of the antibody or antigen-binding portion for the antigen. Such carbohydrate modifications can be accomplished by, for example, altering upstream process technologies, for example, recombinant host cell culture conditions by supplementing the cell culture media with dissolved oxygen (e.g., a dissolved oxygen concentration of 10%, 20%, 30%, 40%, 50%, 60%, 70%, or 80%).

The modulation of the glycosylation of the protein may result in an increase or decrease in the overall amount or level of galactosylation, or galactosylated N-glycans, linked to the recombinant protein. For example, the amount or level of galactosylated N-glycans (e.g., G1F, G1F-GlcNAc, G2F) linked to the protein is increased by about 0.1%, 1%, 1.2%, 1.5%, 2%, 2.2%, 2.5%, 3%, 3.2%, 3.5%, 4%, 4.2%, 4.5%, 5%, 10%, 15%, 20%, 25%, 30%, 35%, 40%, 45% or 50%, 55%, 60%. Ranges within one or more of the preceding values, e.g., about 0.1-5, 0.1-10%, 0.1-15%, 0.1-20%, 0.1-25%, 0.1-30%, 0.1-35%, 0.1-40%, 0.1-45%, 0.1-50%, 0.1-55%, 0.1-60%, 1-5%, 1-10%, 1-15%, 1-20%, 1-25%, 1-30%, 1-35%, 1-40%, 1-45%, 1-50%, 1-55%, 1-60%, 2-5%, 2-10%, 2-15%, 2-20%, 2-25%, 2-30%, 2-35%, 2-40%, 2-45%, 2-50%, 2-55%, 2-60%, 3-5%, 3-10%, 3-15%, 3-20%, 3-25%, 3-30%, 3-35%, 3-40%, 3-45%, 3-50%, 3-55%, 3-60%, 4-5%, 4-10%, 4-15%, 4-20%, 4-25%, 4-30%, 4-35%, 4-40%, 4-45%, 4-50%, 4-55%, 4-60%, 5-10%, 5-15%, 5-20%, 5-25%, 5-30%, 5-35%, 5-40%, 5-45%, 5-50%, 5-55% or 5-60% are contemplated by the invention.

In another embodiment, the modulation of the glycosylation of the recombinant protein results in an increase in the amount or level of G1F and/or G1F-GlcNAc species linked to the protein. For example, the amount or level of G1F and/or G1F-GlcNAc species linked to the recombinant protein is increased by about 0.1%, 1%, 1.2%, 1.5%, 2%, 2.2%, 2.5%, 3%, 3.2%, 3.5%, 4%, 4.2%, 4.5%, 5%, 6%, 7%, 8%, 9%, 10%, 15%, 20%, 25%, 30%, 35%, 40% or 45%. Ranges within one or more of the preceding values, e.g., about 0.1% to 5%, 0.15 to 10%, 1% to 5%, 1% to 10%, 2% to 8%, 3% to 6%, 5% to 8% or 0.1% to 45% are contemplated by the invention.

In another embodiment, the modulation of the glycosylation of the recombinant protein results in an increase in the amount or level of G2F species linked to the recombinant protein. For example, the amount or level of G2F species linked to the protein is increased by about 0.1%, 1%, 1.2%, 1.5%, 2%, 2.2%, 2.5%, 3%, 3.2%, 3.5%, 4%, 4.2%, 4.5%, 5%, 6%, 7%, 8%, 9%, 10% or 15%. Ranges within one or more of the preceding values, e.g., about 0.1% to 5%, 0.1% to 10%, 1% to 5%, 1% to 10%, 2% to 8%, 3% to 6%, 5% to 8% or 0.1% to 15% are contemplated by the invention.

In another embodiment, the modulation of the glycosylation of the protein results in an overall decrease in the amount or level of agalactosyl N-glycan species linked to the recombinant protein. For example, the overall amount or level of agalactosyl N-glycan species linked to the recombinant protein is decreased by about 0.1%, 1%, 1.2%, 1.5%, 2%, 2.2%, 2.5%, 3%, 3.2%, 3.5%, 4%, 4.2%, 4.5%, 5%, 10%, 15%, 20%, 25%, 30%, 35%, 40% or 45%. Ranges within one or more of the preceding values, e.g., about 0.1-5%, 0.1-10%, 0.1-15%, 0.1-20%, 0.1-25%, 0.1-30%, 0.1-35%, 0.1-40%, 0.1-45%, 1-5%, 1-10%, 1-15%, 1-20%, 1-25%, 1-30%, 2-5%, 2-10%, 2-15%, 2-20%, 2-25%, 2-30%, 2-35%, 2-40%, 2-45%, 3-5%, 3-10%, 3-15%, 3-20%, 3-25%, 3-30%, 3-35%, 3-40%, 3-45%, 4-5%, 4-10%, 4-15%, 4-20%, 4-25%, 4-30%, 4-35%, 4-40%, 4-45%, 5-10%, 5-15%, 5-20%, 5-25%, 5-30%, 5-35%, 5-40% or 5-45% are contemplated by the invention.

In another embodiment, the modulation of the glycosylation of the protein results in a decrease in the amount or level of G0, G0-GlcNAc, G0F and/or G0F-GlcNAc species linked to the recombinant protein. For example the amount or level of G0, G0-GlcNAc, G0F and/or G0F-GlcNAc linked to the recombinant protein is decreased by about 0.1%, 1%, 1.2%, 1.5%, 2%, 2.2%, 2.5%, 3%, 3.2%, 3.5%, 4%, 4.2%, 4.5%, 5%, 6%, 7%, 8%, 9%, 10%, 15%, 20%, 25%, 30%, 35%, 40% or 45%. Ranges within one or more of the preceding values, e.g., about 0.1% to 5%, 0.1% to 10%, 0.1% to 20%, 1% to 5%, 1% to 10%, 1% to 20%, 2% to 8%, 3% to 6%, 3% to 20%, 5% to 8%, 5% to 20% or 0.1% to 45%.

In another embodiment, the modulation of the glycosylation of the protein results in an overall increase in the amount or level of agalactosyl N-glycan species linked to the recombinant protein. For example, the overall amount or level of agalactosyl N-glycan species linked to the recombinant protein is increased by about 0.1%, 1%, 1.2%, 1.5%, 2%, 2.2%, 2.5%, 3%, 3.2%, 3.5%, 4%, 4.2%, 4.5%, 5%, 10% or 15%. Ranges within one or more of the preceding values, e.g., about 0.1-2%, 0.1-5%, 0.1-10%, 0.1-15%, 1-2%, 1-5%, 1-10%, 1-15%, 2-5%, 2-10%, 2-15%, 3-5%, 3-10%, 3-15%, 4-10%, 4-15%, 5-10% or 5-15% are contemplated by the invention.

In another embodiment, the modulation of the glycosylation of the protein results in an increase in the amount or level of G0, G0-GlcNAc, G0F and/or G0F-GlcNAc species linked to the recombinant protein. For example, the amount or level of G0, G0-GlcNAc, G0F and/or G0F-GlcNAc linked to the recombinant protein is increased by about 0.1%, 1%, 1.2%, 1.5%, 2%, 2.2%, 2.5%, 3%, 3.2%, 3.5%, 4%, 4.2%, 4.5%, 5%, 10% or 15%. Ranges within one or more of the preceding values, e.g., about 0.1-2%, 0.1-5%, 0.1-10%, 0.1-15%, 1-2%, 1-5%, 1-10%, 1-15%, 2-5%, 2-10%, 2-15%, 3-5%, 3-10%, 3-15%, 4-10%, 4-15%, 5-10% or 5-15%.

In another embodiment, the modulation of the glycosylation of the protein (e.g., antibody or DVD-Ig) results in an increase or decrease in the overall amount or level of mannosylation in the recombinant protein. For example, the amount or level of mannosylation is increased or decreased by about 0.1%, 1%, 1.2%, 1.5%, 2%, 2.2%, 2.5%, 2.55%, 3%, 3.2%, 3.5%, 4%, 4.2%, 4.5%, 5%, 10%, 15%, 20%, 25% or 30%. Ranges within one or more of the preceding values, e.g., about 0.1-5%, 0.1-10%, 0.1-15%, 0.1-20%, 0.1-25%, 0.1-30%, 1-5%, 1-10%, 1-15%, 1-20%, 1-25%, 1-30%, 2-5%, 2-10%, 2-15%, 2-20%, 2-25%, 2-30%, 3-5%, 3-10%, 3-15%, 3-20%, 3-25%, 3-30%, 4-5%, 4-10%, 4-15%, 4-20%, 4-25% or 4-30% are contemplated by the invention.

In another embodiment, the increase in mannosylation in the recombinant protein comprises an increase in the amount or level of a high mannose N-glycan oligosaccharide. A high-mannose N-glycan has more than one mannose linked to the non-reducing terminal of the core structure. For example, the high mannose N-glycan oligosaccharide is selected from the group consisting of Man 5 glycan, Man 6 glycan, Man 7 glycan and Man 8 glycan. In one embodiment, the amount or level of at least one of Man 5 glycan, Man 6 glycan, Man 7 glycan and/or Man 8 glycan is increased by about 0.1%, 1%, 1.2%, 1.5%, 2%, 2.2%, 2.5%, 3%, 3.2%, 3.5%, 4%, 4.2%, 4.5%, 5%, 10%, 15%, 20%, 25%, or 30%. Ranges within one or more of the preceding values, e.g., about 0.1-5%, 0.1-10%, 0.1-15%, 0.1-20%, 0.1-25%, 0.1-30%, 1-5%, 1-10%, 1-15%, 1-20%, 1-25%, 1-30%, 2-5%, 2-10%, 2-15%, 2-20%, 2-25%, 2-30%, 3-5%, 3-10%, 3-15%, 3-20%, 3-25%, 3-30%, 4-5%, 4-10%, 4-15%, 4-20%, 4-25% or 4-30% are contemplated by the invention.

In another embodiment, the decrease in mannosylation in the recombinant protein comprises a decrease in the amount or level of a high mannose N-glycan oligosaccharide. In one embodiment, the amount or level of at least one of Man 5 glycan, Man 6 glycan, Man 7 glycan and/or Man 8 glycan is decreased by about 0.1%, 1%, 1.2%, 1.5%, 2%, 2.2%, 2.5%, 2.55%, 3%, 3.2%, 3.5%, 4%, 4.2%, 4.5%, 5%, 10%, 15%, 20%, 25%, or 30%. Ranges within one or more of the preceding values, e.g., about 0.1-3%, 0.1-5%, 0.1-10%, 0.1-15%, 0.1-20%, 0.1-25%, 0.1-30%, 1-5%, 1-10%, 1-15%, 1-20%, 1-25%, 1-30%, 2-5%, 2-10%, 2-15%, 2-20%, 2-25%, 2-30%, 3-5%, 3-10%, 3-15%, 3-20%, 3-25%, 3-30%, 4-5%, 4-10%, 4-15%, 4-20%, 4-25% or 4-30% are contemplated by the invention.

It is known to those skilled in the art that differing protein glycosylation profiles may result in differing protein characteristics. For instance, the efficacy of a therapeutic protein produced in a microorganism host, such as yeast, and glycosylated utilizing the yeast endogenous pathway may be reduced compared to that of the same protein expressed in a mammalian cell, such as a CHO cell line. Such glycoproteins may also be immunogenic in humans and show reduced half-life in vivo after administration. Specific receptors in humans and other animals may recognize specific glycosyl residues and promote the rapid clearance of the protein from the bloodstream. Other adverse effects may include changes in protein folding, solubility, susceptibility to proteases, trafficking, transport, compartmentalization, secretion, recognition by other proteins or factors, antigenicity, or allergenicity. Accordingly, using the methods of the invention, one of skill in the art may modulate the glycosylation profile of a protein, e.g., an antibody or DVD-Ig, to achieve a desired activity such as increased or decreased rate of clearance and/or increased ADCC activity.

### Upstream Process Technologies

The methods of the present invention may be used to produce a recombinant protein (e.g., an antibody, or antigen binding fragment thereof, or a DVD-Ig) with a modulated glycosylation profile. In one embodiment, the methods of the invention involve modification of the conditions used during upstream protein production, such as recombinant cell culture conditions. For example, the methods of the invention comprise supplementing the recombinant cell culture media with dissolved oxygen (e.g., dissolved oxygen at a concentration of 10%, 20%, 30%, 40%, 50%, 60%, 70%, or 80%) to modulate the glycosylation profile of the protein.

The upstream process technologies may be used alone or in combination with the downstream process technologies described below.

In one embodiment, the methods described herein produce a recombinant protein with a modulated glycosylation profile wherein the overall galactosylation level resulting from the modulation of any one of the galactosylated glycan species such as G1F, G1F-GlcNAc and/or G2F, is increased by about 0.1%, 1%, 1.2%, 1.5%, 2%, 2.2%, 2.5%, 3%, 3.2%, 3.5%, 4%, 4.2%, 4.5%, 5%, 10%, 15%, 20%, 25%, 30%, 35%, 40%, 45% or 50%, 55% or 60%, and ranges within one or more of the preceding. In one aspect of this embodiment, the overall galactosylation level is increased by about 0.1-5%, 0.1-10%, 0.1-15%, 0.1-20%, 0.1-25%, 0.1-30%, 0.1-35%, 0.1-40%, 0.1-45%, 0.1-50%, 0.1-55%, 0.1-60%, 1-5%, %, 1-10%, 1-15%, 1-20%, 1-25%, 1-30%, 1-35%, 1-40%, 1-45%, 1-50%, 1-55%, 1-60%, 2-5%, 2-10%, 2-15%, 2-20%, 2-25%, 2-30%, 2-35%, 2-40%, 2-45%, 2-50%, 2-55%, 2-60%, 3-5%, 3-10%, 3-15%, 3-20%, 3-25%, 3-30%, 3-35%, 3-40%, 3-45%, 3-50%, 3-55%, 3-60%, 4-5%, 4-10%, 4-15%, 4-20%, 4-25%, 4-30%, 4-35%, 4-40%, 4-45%, 4-50%, 4-55%, 4-60%, 5-10%, 5-15%, 5-20%, 5-25%, 5-30%, 5-35%, 5-40%, 5-45%, 5-50%, 5-55% or 5-60% and ranges within one or more of the preceding.

In another embodiment, the methods described herein produce a protein with a modulated glycosylation profile wherein the overall mannosylation level resulting from the modulation of any one of the high mannose N-glycan oligosaccharides, such as Man 5 glycan, Man 6 glycan, Man 7 glycan or Man 8 glycan, is increased by about 0.1%, 1%, 1.2%, 1.5%, 2%, 2.2%, 2.5%, 3%, 3.2%, 3.5%, 4%, 4.2%, 4.5%, 5%, 10%, 15%, 20%, 25%, or 30%, and ranges within one or more of the preceding. In one aspect of this embodiment, the overall high mannose N-glycan level is increased by about 0.1-5%, 0.1-10%, 0.1-15%, 0.1-20%, 0.1-25%, 0.1-30%, 1-5%, 1-10%, 1-15%, 1-20%, 1-25%, 1-30%, 2-5%, 2-10%, 2-15%, 2-20%, 2-25%, 2-30%, 3-5%, 3-10%, 3-15%, 3-20%, 3-25%, 3-30%, 4-5%, 4-10%, 4-15%, 4-20%, 4-25% or 4-30%, and ranges within one or more of the preceding.

In another embodiment, the methods described herein produce a protein with a modulated glycosylation profile wherein the overall mannosylation level resulting from the modulation of any one of the high mannose N-glycan oligosaccharides, such as Man 5 glycan, Man 6 glycan, Man 7 glycan or Man 8 glycan, is decreased by about 0.1%, 1%, 1.2%, 1.5%, 2%, 2.2%, 2.5%, 2.55%, 3%, 3.2%, 3.5%, 4%, 4.2%, 4.5%, 5%, 10%, 15%, 20%, 25%, or 30%, and ranges within one or more of the preceding. In one aspect of this embodiment, the overall high mannose N-glycan level is decreased by about 0.1-3%, 0.1-5%, 0.1-10%, 0.1-15%, 0.1-20%, 0.1-25%, 0.1-30%, 1-5%, 1-10%, 1-15%, 1-20%, 1-25%, 1-30%, 2-5%, 2-10%, 2-15%, 2-20%, 2-25%, 2-30%, 3-5%, 3-10%, 3-15%, 3-20%, 3-25%, 3-30%, 4-5%, 4-10%, 4-15%, 4-20%, 4-25% or 4-30%, and ranges within one or more of the preceding.

In another embodiment, the methods described herein produce a protein with a modulated glycosylation profile wherein the overall agalactosyl N-glycan level resulting from the modulation of any one of the agalactosyl N-glycans, such as G0, G0-GlcNAc, G0F, G0F-GlcNAc, is decreased by about 0.1%, 1%, 1.2%, 1.5%, 2%, 2.2%, 2.5%, 3%, 3.2%, 3.5%, 4%, 4.2%, 4.5%, 5%, 10%, 15%, 20%, 25%, 30%, 35%, 40% or 45%, and ranges within one or more of the preceding. In one aspect of this embodiment, the overall agalactosyl N-glycans level is decreased by about 0.1-5%, 0.1-10%, 0.1-15%, 0.1-20%, 0.1-25%, 0.1-30%, 0.1-35%, 0.1-40%, 0.1-45%, 1-5%, 1-10%, 1-15%, 1-20%, 1-25%, 1-30%, 2-5%, 2-10%, 2-15%, 2-20%, 2-25%, 2-30%, 2-35%, 2-40%, 2-45%, 3-5%, 3-10%, 3-15%, 3-20%, 3-25%, 3-30%, 3-35%, 3-40%, 3-45%, 4-5%, 4-10%, 4-15%, 4-20%, 4-25%, 4-30%, 4-35%, 4-40%, 4-45%, 5-10%, 5-15%, 5-20%, 5-25%, 5-30%, 5-35%, 5-40% or 5-45%, and ranges within one or more of the preceding.

In another embodiment, the methods described herein produce a protein with a modulated glycosylation profile wherein the overall agalactosyl N-glycan level resulting from the modulation of any one of the agalactosyl N-glycans, such as G0, G0-GlcNAc, G0F, G0F-GlcNAc, is increased by about 0.1%, 1%, 1.2%, 1.5%, 2%, 2.2%, 2.5%, 3%, 3.2%, 3.5%, 4%, 4.2%, 4.5%, 5%, 10% or 15%, and ranges within one or more of the preceding. In one aspect of this embodiment, the overall agalactosyl N-glycans level is increased by about 0.1-2%, 0.1-5%, 0.1-10%, 0.1-15%, 1-2%, 1-5%, 1-10%, 1-15%, 2-5%, 2-10%, 2-15%, 3-5%, 3-10%, 3-15%, 4-10%, 4-15%, 5-10% or 5-15%, and ranges within one or more of the preceding.

As described herein, the host cell culture conditions can be modified as compared to conditions during production of the same protein without modulation of the glycosylation profile. In one embodiment, a protein with a modulated glycosylation profile is produced by culturing cells expressing the antibody, or antigen binding fragment thereof, in cell culture media supplemented with a certain dissolved oxygen concentration (e.g., 10%, 20%, 30%, 40%, 50%, 60%, 70%, or 80%).

To express a protein with a modulated glycosylation profile (e.g., an antibody, such as for example, adalimumab, or an antigen-binding fragment thereof), DNAs encoding the protein, such as DNAs encoding partial or full-length light and heavy chains in the case of antibodies, are inserted into one or more expression vector such that the genes are operatively linked to transcriptional and translational control sequences. (See, e.g., U.S. Pat. No. 6,090,382, the entire contents of which are incorporated herein by reference.) In this context, the term “operatively linked” is intended to mean that a gene encoding the protein is ligated into a vector such that transcriptional and translational control sequences within the vector serve their intended function of regulating the transcription and translation of the gene. The expression vector and expression control sequences are chosen to be compatible with the expression host cell used. In certain embodiments, the protein with a modulated glycosylation profile will comprising multiple polypeptides, such as the heavy and light chains of an antibody. Thus, in certain embodiments, genes encoding multiple polypeptides, such as antibody light chain genes and antibody heavy chain genes, can be inserted into a separate vector or, more typically, the genes are inserted into the same expression vector. Genes are inserted into expression vectors by standard methods (e.g., ligation of complementary restriction sites on the gene fragment and vector, or blunt end ligation if no restriction sites are present). Prior to insertion of the gene or genes, the expression vector may already carry additional polypeptide sequences, such as, but not limited to, antibody constant region sequences. For example, one approach to converting the anti-TNFα antibody or anti-TNFα antibody-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the protein from a host cell. The gene can be cloned into the vector such that the signal peptide is linked in-frame to the amino terminus of the gene. The signal peptide can be an immunoglobulin signal peptide or a heterologous signal peptide (i.e., a signal peptide from a non-immunoglobulin protein).

In addition to protein coding genes, a recombinant expression vector can carry one or more regulatory sequence that controls the expression of the protein coding genes in a host cell. The term “regulatory sequence” is intended to include promoters, enhancers and other expression control elements (e.g., polyadenylation signals) that control the transcription or translation of the protein coding genes. Such regulatory sequences are described, e.g., in Goeddel; Gene Expression Technology: Methods in Enzymology 185, Academic Press, San Diego, Calif. (1990), the entire teaching of which is incorporated herein by reference. It will be appreciated by those skilled in the art that the design of the expression vector, including the selection of regulatory sequences may depend on such factors as the choice of the host cell to be transformed, the level of expression of protein desired, etc. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see, e.g., U.S. Pat. No. 5,168,062 by Stinski, U.S. Pat. No. 4,510,245 by Bell et al. and U.S. Pat. No. 4,968,615 by Schaffner et al., the entire teachings of which are incorporated herein by reference.

A recombinant expression vector may also carry one or more additional sequences, such as a sequence that regulates replication of the vector in host cells (e.g., origins of replication) and/or a selectable marker gene. The selectable marker gene facilitates selection of host cells into which the vector has been introduced (see e.g., U.S. Pat. Nos. 4,399,216, 4,634,665 and 5,179,017, all by Axel et al., the entire teachings of which are incorporated herein by reference). For example, typically the selectable marker gene confers resistance to drugs, such as G418, hygromycin or methotrexate, on a host cell into which the vector has been introduced. Suitable selectable marker genes include the dihydrofolate reductase (DHFR) gene (for use in dhfr-host cells with methotrexate selection/amplification) and the neo gene (for G418 selection).

An antibody, or antigen binding fragment thereof, to be used in the method of preparing a protein with a modulated glycosylation profile can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel et al. (eds.) Current Protocols in Molecular Biology, Greene Publishing Associates, (1989) and in U.S. Pat. Nos. 4,816,397 & 6,914,128, the entire teachings of which are incorporated herein.

For expression of a protein, for example, the light and heavy chains of an antibody, the expression vector(s) encoding the protein is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the proteins of the invention in either prokaryotic or eukaryotic host cells, expression of antibodies in eukaryotic cells, such as mammalian host cells, is suitable because such eukaryotic cells, and in particular mammalian cells, are more likely than prokaryotic cells to assemble and secrete a properly folded and immunologically active protein. Prokaryotic expression of protein genes has been reported to be ineffective for production of high yields of active protein (Boss and Wood (1985) Immunology Today 6:12-13, the entire teaching of which is incorporated herein by reference).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, e.g., Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One suitable E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for polypeptide encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of proteins with modulated glycosylation profiles, for example, glycosylated antibodies, are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

Mammalian cells can be used for expression and production of the protein compositions of the invention, however other eukaryotic cell types can also be employed in the context of the instant invention. See, e.g., Winnacker, From Genes to Clones, VCH Publishers, N.Y., N.Y. (1987). Suitable mammalian host cells for expressing recombinant proteins according to the invention include Chinese Hamster Ovary (CHO cells) (including dhfr-CHO cells, described in Urlaub and Chasin, (1980) PNAS USA 77:4216-4220, used with a DHFR selectable marker, e.g., as described in Kaufman and Sharp (1982) Mol. Biol. 159:601-621, the entire teachings of which are incorporated herein by reference), NS0 myeloma cells, COS cells and SP2 cells. When recombinant expression vectors encoding protein genes are introduced into mammalian host cells, the antibodies are produced by culturing the host cells for a period of time sufficient to allow for expression of the antibody in the host cells or secretion of the antibody into the culture medium in which the host cells are grown. Other examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), the entire teachings of which are incorporated herein by reference.

Host cells are transformed with the above-described expression or cloning vectors for protein production and cultured in conventional nutrient media modified as appropriate for inducing promoters, selecting transformants, or amplifying the genes encoding the desired sequences.

The host cells used to produce a protein may be cultured in a variety of media which are supplemented in accordance with the present invention. Commercially available media such as Ham's F10™ (Sigma), Minimal Essential Medium™ (MEM), (Sigma), RPMI-1640 (Sigma), and Dulbecco's Modified Eagle's Medium™ (DMEM), (Sigma), Iscove's Modified Dulbecco's Medium, Minimal Essential Medium-alpha. (MEM-alpha), DME/F12, alpha MEM, Basal Medium Eagle with Earle's BSS, DMEM high Glucose, with L-Glutamine, DMEM high glucose, without L-Glutamine, DMEM low Glucose, without L-Glutamine, DMEM:F12 1:1, with L-Glutamine, GMEM (Glasgow's MEM), GMEM with L-glutamine, Grace's Complete Insect Medium, Grace's Insect Medium, without FBS, Ham's F-10, with L-Glutamine, Ham's F-12, with L-Glutamine, IMDM with HEPES and L-Glutamine, IMDM with HEPES and without L-Glutamine, IPL-41 Insect Medium, L-15 (Leibovitz)(2.times.), without L-Glutamine or Phenol Red, L-15 (Leibovitz), without L-Glutamine, McCoy's 5A Modified Medium, Medium 199, MEM Eagle, without L-Glutamine or Phenol Red (2.times.), MEM Eagle-Earle's BSS, with L-glutamine, MEM Eagle-Earle's BSS, without L-Glutamine, MEM Eagle-Hanks BSS, without L-Glutamine, NCTC-109, with L-Glutamine, Richter's CM Medium, with L-Glutamine, RPMI 1640 with HEPES, L-Glutamine and/or Penicillin-Streptomycin, RPMI 1640, with L-Glutamine, RPMI 1640, without L-Glutamine, Schneider's Insect Medium are suitable for culturing host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells, the entire teachings of which are incorporated herein by reference.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Host cells can also be used to produce portions of intact proteins, for example, antibodies, including Fab fragments or scFv molecules. It is understood that variations on the above procedure are within the scope of the present invention. For example, in certain embodiments it may be desirable to transfect a host cell with DNA encoding either the light chain or the heavy chain (but not both) of an antibody. Recombinant DNA technology may also be used to remove some or all of the DNA encoding either or both of the light and heavy chains that is not necessary for binding to an antigen. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the target antibody, depending on the specificity of the antibody of the invention, by crosslinking an antibody of the invention to a second antibody by standard chemical crosslinking methods.

In a suitable system for recombinant expression of a protein, for example, an antibody, or antigen-binding portion thereof, or a DVD-Ig, a recombinant expression vector encoding the protein, for example, both an antibody heavy chain and an antibody light chain, is introduced into dhfr-CHO cells by calcium phosphate-mediated transfection. Within the recombinant expression vector, the protein gene(s) are each operatively linked to CMV enhancer/AdMLP promoter regulatory elements to drive high levels of transcription of the gene(s). The recombinant expression vector also carries a DHFR gene, which allows for selection of CHO cells that have been transfected with the vector using methotrexate selection/amplification. The selected transformant host cells are cultured to allow for expression of the protein, for example, the antibody heavy and light chains, and intact protein, for example, an antibody, is recovered from the culture medium. Standard molecular biology techniques are used to prepare the recombinant expression vector, transfect the host cells, select for transformants, culture the host cells and recover the protein from the culture medium.

When using recombinant techniques, the protein, for example, antibodies or antigen binding fragments thereof, can be produced intracellularly, in the periplasmic space, or directly secreted into the medium. In one aspect, if the protein is produced intracellularly, as a first step, the particulate debris, either host cells or lysed cells (e.g., resulting from homogenization), can be removed, e.g., by centrifugation or ultrafiltration. Where the protein is secreted into the medium, supernatants from such expression systems can be first concentrated using a commercially available protein concentration filter, e.g., an Amicon™ or Millipore Pellicon™ ultrafiltration unit.

Some antibodies can be secreted directly from the cell into the surrounding growth media; others are made intracellularly. For antibodies made intracellularly, the first step of a purification process typically involves: lysis of the cell, which can be done by a variety of methods, including mechanical shear, osmotic shock, or enzymatic treatments. Such disruption releases the entire contents of the cell into the homogenate, and in addition produces subcellular fragments that are difficult to remove due to their small size. These are generally removed by differential centrifugation or by filtration. Where the antibody is secreted, supernatants from such expression systems are generally first concentrated using a commercially available protein concentration filter, e.g., an Amicon™ or Millipore Pellicon™ ultrafiltration unit. Where the antibody is secreted into the medium, the recombinant host cells can also be separated from the cell culture medium, e.g., by tangential flow filtration. Antibodies can be further recovered from the culture medium using the antibody purification methods of the invention.

In accordance with the present invention, modulation of the glycosylation profile of the protein (e.g., antibody such as adalimumab or DVD-Ig) produced by recombinant cell culture can be achieved by supplementing the cell culture media with a dissolved oxygen concentration (e.g., 10%, 20%, 30%, 40%, 50%, 60%, 70%, 80%) Specific host cell culture conditions can be used with various cultivation methods including, but not limited to, batch, fed-batch, chemostat and perfusion, and with various cell culture equipment including, but not limited to, shake flasks with or without suitable agitation, spinner flasks, stirred bioreactors, airlift bioreactors, membrane bioreactors, reactors with cells retained on a solid support or immobilized/entrapped as in microporous beads, and any other configuration appropriate for optimal growth and productivity of the desired host cell line.

Supplementation with Dissolved Oxygen to Modulate the Glycosylation Profile of the Expressed Protein

The present invention relates to methods for modulating the glycosylation profile of a recombinant protein by supplementing cell culture media used to produce the protein with dissolved oxygen. In the present invention dissolved oxygen is used to affect the glycosylation profile of the recombinant protein. For example, but not by way of limitation, by adjusting the concentration of dissolved oxygen the glycosylation profile can be modulated.

In certain embodiments, a protein with a modulated glycosylation profile is prepared by supplementation of cell culture media with a certain dissolved oxygen concentration (e.g., 10%, 20%, 30%, 40%, 50%, 60%, 70%, or 80%).

In certain embodiments, the cell culture media is supplemented with different concentrations of dissolved oxygen at different cell culture temperatures to modulate the glycosylation profile of the protein such that the overall galactosylation amount or level, increases or decreases by about 0.1%, 0.7%, 0.9%, 1%, 1.2%, 1.5%, 2%, 2.2%, 2.5%, 3%, 3.2%, 3.5%, 4%, 4.2%, 4.5%, 5%, 7%, 10%, 15%, 20%, 25%, 30%, 35%, 40%, 45% or 50%, 55% or 60%.

In certain embodiments, the cell culture media is supplemented with dissolved oxygen in an amount effective to modulate the glycosylation profile of the protein such that the overall galactosylation amount or level, resulting from the modulation of at least one of the galactosylated glycan species such as G1F, G1F-GlcNAc and/or G2F, is increased by about 0.1%, 1%, 1.2%, 1.5%, 2%, 2.2%, 2.5%, 3%, 3.2%, 3.5%, 4%, 4.2%, 4.5%, 5%, 10%, 15%, 20%, 25%, 30%, 35%, 40%, 45% or 50%, 55% or 60%, and ranges within one or more of the preceding. In one aspect of this embodiment, the overall galactosylation amount or level is increased by about 0.1-5%, 0.1-10%, 0.1-15%, 0.1-20%, 0.1-25%, 0.1-30%, 0.1-35%, 0.1-40%, 0.1-45%, 0.1-50%, 0.1-55%, 0.1-60%, 1-5%, %, 1-10%, 1-15%, 1-20%, 1-25%, 1-30%, 1-35%, 1-40%, 1-45%, 1-50%, 1-55%, 1-60%, 2-5%, 2-10%, 2-15%, 2-20%, 2-25%, 2-30%, 2-35%, 2-40%, 2-45%, 2-50%, 2-55%, 2-60%, 3-5%, 3-10%, 3-15%, 3-20%, 3-25%, 3-30%, 3-35%, 3-40%, 3-45%, 3-50%, 3-55%, 3-60%, 4-5%, 4-10%, 4-15%, 4-20%, 4-25%, 4-30%, 4-35%, 4-40%, 4-45%, 4-50%, 4-55%, 4-60%, 5-10%, 5-15%, 5-20%, 5-25%, 5-30%, 5-35%, 5-40%, 5-45%, 5-50%, 5-55% or 5-60%, and ranges within one or more of the preceding.

In certain embodiments, the cell culture media is supplemented with dissolved oxygen in an amount effective to modulate the glycosylation profile of the protein such that the overall mannosylation amount or level, resulting from the modulation of at least one of the high mannose N-glycan oligosaccharides, such as Man 5 glycan, Man 6 glycan, Man 7 glycan or Man 8 glycan, is increased by about 0.1%, 1%, 1.2%, 1.5%, 2%, 2.2%, 2.5%, 3%, 3.2%, 3.5%, 4%, 4.2%, 4.5%, 5%, 10%, 15%, 20%, 25%, or 30%, and ranges within one or more of the preceding. In one aspect of this embodiment, the overall high mannose N-glycan amount or level is increased by about 0.1-5%, 0.1-10%, 0.1-15%, 0.1-20%, 0.1-25%, 0.1-30%, 1-5%, 1-10%, 1-15%, 1-20%, 1-25%, 1-30%, 2-5%, 2-10%, 2-15%, 2-20%, 2-25%, 2-30%, 3-5%, 3-10%, 3-15%, 3-20%, 3-25%, 3-30%, 4-5%, 4-10%, 4-15%, 4-20%, 4-25% or 4-30%, and ranges within one or more of the preceding.

In certain embodiments, the cell culture media is supplemented with dissolved oxygen in an amount effective to modulate the glycosylation profile of the protein such that the overall mannosylation amount or level, resulting from the modulation of at least one of the high mannose N-glycan oligosaccharides, such as Man 5 glycan, Man 6 glycan, Man 7 glycan or Man 8 glycan, is decreased by about 0.1%, 1%, 1.2%, 1.5%, 2%, 2.2%, 2.5%, 2.55%, 3%, 3.2%, 3.5%, 4%, 4.2%, 4.5%, 5%, 10%, 15%, 20%, 25%, or 30%, and ranges within one or more of the preceding. In one aspect of this embodiment, the overall high mannose N-glycan amount or level is decreased by about 0.1-3%, 0.1-5%, 0.1-10%, 0.1-15%, 0.1-20%, 0.1-25%, 0.1-30%, 1-5%, 1-10%, 1-15%, 1-20%, 1-25%, 1-30%, 2-5%, 2-10%, 2-15%, 2-20%, 2-25%, 2-30%, 3-5%, 3-10%, 3-15%, 3-20%, 3-25%, 3-30%, 4-5%, 4-10%, 4-15%, 4-20%, 4-25% or 4-30%, and ranges within one or more of the preceding.

In certain embodiments, the cell culture media is supplemented with dissolved oxygen in an amount effective to modulate the glycosylation profile of the protein such that the overall agalactosyl N-glycan level resulting from the modulation of at least one of the agalactosyl N-glycans, such as G0, G0-GlcNAc, G0F, G0F-GlcNAc, is decreased by about 0.1%, 1%, 1.2%, 1.5%, 2%, 2.2%, 2.5%, 3%, 3.2%, 3.5%, 4%, 4.2%, 4.5%, 5%, 10%, 15%, 20%, 25%, 30%, 35%, 40% or 45%, and ranges within one or more of the preceding. In one aspect of this embodiment, the overall agalactosyl N-glycans level is decreased by about 0.1-5%, 0.1-10%, 0.1-15%, 0.1-20%, 0.1-25%, 0.1-30%, 0.1-35%, 0.1-40%, 0.1-45%, 1-5%, 1-10%, 1-15%, 1-20%, 1-25%, 1-30%, 2-5%, 2-10%, 2-15%, 2-20%, 2-25%, 2-30%, 2-35%, 2-40%, 2-45%, 3-5%, 3-10%, 3-15%, 3-20%, 3-25%, 3-30%, 3-35%, 3-40%, 3-45%, 4-5%, 4-10%, 4-15%, 4-20%, 4-25%, 4-30%, 4-35%, 4-40%, 4-45%, 5-10%, 5-15%, 5-20%, 5-25%, 5-30%, 5-35%, 5-40% or 5-45%, and ranges within one or more of the preceding.

In certain embodiments, the cell culture media is supplemented with dissolved oxygen in an amount effective to modulate the glycosylation profile of the protein such that the overall agalactosyl N-glycan level resulting from the modulation of any one of the agalactosyl N-glycans, such as G0, G0-GlcNAc, G0F, G0F-GlcNAc, is increased by about 0.1%, 1%, 1.2%, 1.5%, 2%, 2.2%, 2.5%, 3%, 3.2%, 3.5%, 4%, 4.2%, 4.5%, 5%, 10% or 15%, and ranges within one or more of the preceding. In one aspect of this embodiment, the overall agalactosyl N-glycans level is increased by about 0.1-2%, 0.1-5%, 0.1-10%, 0.1-15%, 1-2%, 1-5%, 1-10%, 1-15%, 2-5%, 2-10%, 2-15%, 3-5%, 3-10%, 3-15%, 4-10%, 4-15%, 5-10% or 5-15%, and ranges within one or more of the preceding. In certain embodiments, the cell culture media is supplemented, for example, at the start of culture, or in a fed-batch or in a continuous manner. The feed amounts may be calculated to achieve a certain concentration based on off-line or on-line measurements. The addition of one or more supplements may be based on measured glycosylation profiles. The resulting media can be used in various cultivation methods including, but not limited to, batch, fed-batch, chemostat and perfusion, and with various cell culture equipment including, but not limited to, shake flasks with or without suitable agitation, spinner flasks, stirred bioreactors, airlift bioreactors, membrane bioreactors, reactors with cells retained on a solid support or immobilized/entrapped as in microporous beads, incubation vessels, microtiter plates, capillaries, multi-well plates and any other configuration appropriate for optimal growth and productivity of the desired host cell line. Additional cell culture equipment may be used such as fermentor tanks, air lifts, culture flasks, spinner flasks, microcarriers, fluidized beds, hollow fibers, roller bottles or packed beds. In addition, the harvest criterion for these cultures may be chosen, for example, based on choice of harvest viability or culture duration, to further optimize a certain targeted glycosylation profiles.

### Down Stream Process Technologies

The protein compositions of the invention may be purified using downstream process technologies (e.g., purification or concentration), following production using the upstream process technologies of the present invention. For example, once a clarified solution or mixture comprising the protein with a modulated glycosylation profile, e.g., an antibody or DVD-Ig, has been obtained, separation of the protein from process-related impurities, such as the other proteins produced by the host cell, as well as product-related substances, such acidic or basic variants, is performed. In certain embodiments, the initial steps of the purification methods involve the clarification and primary recovery of an antibody or DVD-Ig from a sample matrix by methods such as centrifugation, depth filtration and/or viral inactivation/reduction. In certain non-limiting embodiments, further separation is performed using cation exchange chromatography, anion exchange chromatography, and/or multi-mode chromatography. In certain embodiments, a combination of one or more different purification techniques, including affinity separation step(s), ion exchange separation step(s), mixed-mode step(s), and/or hydrophobic interaction separation step(s) can also be employed. Such additional purification steps separate mixtures of proteins on the basis of their charge, degree of hydrophobicity, and/or size. Continuous and recycle chromatography are also applicable to chromatography methods where the protein with a modulated glycosylation profile is collected in the unbound faction during chromatography or where the protein is first bound to the chromatography resin and subsequently recovered by washing the media with conditions that elute the bound component. Numerous chromatography resins are commercially available for each of these techniques, allowing accurate tailoring of the purification scheme to the particular protein involved. Each of the separation methods allow proteins to either traverse at different rates through a column, achieving a physical separation that increases as they pass further through the column, or to adhere selectively to a separation resin (or medium). The proteins are then differentially eluted using different eluents. In some cases, the protein with a modulated glycosylation profile is separated from impurities when the impurities specifically adhere to the column's resin and the protein does not, i.e., the protein is contained in the effluent, while in other cases the protein will adhere to the column's resin, while the impurities and/or product-related substances are extruded from the column's resin during a wash cycle. Following chromatographic polishing steps the protein compositions of the invention may be further purified using viral filtration. Ultrafiltration and/or diafiltration may be used to further concentrate and formulate the protein, e.g., an antibody or DVD-Ig product.

The glycosylation profile of the protein prepared by the methods of the invention can be analyzed using methods well known to those skilled in the art, e.g., removal and derivatization of N-glycans followed by NP-HPLC analysis, weak cation exchange chromatography (WCX), capillary isoelectric focusing (cIEF), size-exclusion chromatography, Poros A HPLC Assay, Host cell Protein ELISA, DNA assay, and western blot analysis.

### III. Methods of Treatment Using Recombinant Proteins with Modulated Glycosylation Profiles of the Invention

The compositions comprising a recombinant protein with a modulated glycosylation profile, for example a recombinant protein such as an antibody, e.g., adalimumab, with an increased galactosylation level or amount, an increased or decreased mannosylation level or amount and/or an increased or decreased level or amount of agalactosyl N-glycans, of the invention may be used to treat any disorder in a subject for which the therapeutic protein (e.g., an antibody, such as adalimumab) comprised in the composition is appropriate for treating.

A “disorder” is any condition that would benefit from treatment with the therapeutic protein with a modulated glycosylation profile. This includes chronic and acute disorders or diseases including those pathological conditions which predispose the subject to the disorder in question. In the case of an anti-TNFα antibody, or antigen binding fragment thereof, such as adalimumab, a therapeutically effective amount of the composition comprising a protein with a modulated glycosylation profile may be administered to treat a disorder in which TNFα activity is detrimental.

A disorder in which TNFα activity is detrimental includes a disorder in which inhibition of TNFα activity is expected to alleviate the symptoms and/or progression of the disorder. Such disorders may be evidenced, for example, by an increase in the concentration of TNFα in a biological fluid of a subject suffering from the disorder (e.g., an increase in the concentration of TNFα in serum, plasma, synovial fluid, etc. of the subject), which can be detected, for example, using an anti-TNFα antibody.

TNFα has been implicated in the pathophysiology of a wide variety of a TNFα-related disorders including sepsis, infections, autoimmune diseases, transplant rejection and graft-versus-host disease (see e.g., Moeller, A., et al. (1990) Cytokine 2:162-169; U.S. Pat. No. 5,231,024 to Moeller et al.; European Patent Publication No. 260 610 B1 by Moeller, A., et al. Vasilli, P. (1992) Annu. Rev. Immunol. 10:411-452; Tracey, K. J. and Cerami, A. (1994) Annu. Rev. Med. 45:491-503). Accordingly, the protein with a modulated glycosylation profile of the invention may be used to treat an autoimmune disease, such as rheumatoid arthritis, juvenile idiopathic arthritis, or psoriatic arthritis, an intestinal disorder, such as Crohn's disease or ulcerative colitis, a spondyloarthropathy, such as ankylosing spondylitis, or a skin disorder, such as psoriasis.

Disorders in which TNFα activity is detrimental are well known in the art and described in detail in U.S. Pat. No. 8,231,876 and U.S. Pat. No. 6,090,382, the entire contents of each of which are expressly incorporated herein by reference. In one embodiment, “a disorder in which TNFα activity is detrimental” includes sepsis (including septic shock, endotoxic shock, gram negative sepsis and toxic shock syndrome), autoimmune diseases (including rheumatoid arthritis, rheumatoid spondylitis, osteoarthritis and gouty arthritis, allergy, multiple sclerosis, autoimmune diabetes, autoimmune uveitis, nephrotic syndrome, multisystem autoimmune diseases, lupus (including systemic lupus, lupus nephritis and lupus cerebritis), Crohn's disease and autoimmune hearing loss), infectious diseases (including malaria, meningitis, acquired immune deficiency syndrome (AIDS), influenza and cachexia secondary to infection), allograft rejection and graft versus host disease, malignancy, pulmonary disorders (including adult respiratory distress syndrome (ARDS), shock lung, chronic pulmonary inflammatory disease, pulmonary sarcoidosis, pulmonary fibrosis, silicosis, idiopathic interstitial lung disease and chronic obstructive airway disorders (COPD), such as asthma), intestinal disorders (including inflammatory bowel disorders, idiopathic inflammatory bowel disease, Crohn's disease and Crohn's disease-related disorders (including fistulas in the bladder, vagina, and skin; bowel obstructions; abscesses; nutritional deficiencies; complications from corticosteroid use; inflammation of the joints; erythem nodosum; pyoderma gangrenosum; lesions of the eye, Crohn's related arthralgias, fistulizing Crohn's indeterminant colitis and pouchitis), cardiac disorders (including ischemia of the heart, heart insufficiency, restenosis, congestive heart failure, coronary artery disease, angina pectoris, myocardial infarction, cardiovascular tissue damage caused by cardiac arrest, cardiovascular tissue damage caused by cardiac bypass, cardiogenic shock, and hypertension, atherosclerosis, cardiomyopathy, coronary artery spasm, coronary artery disease, valvular disease, arrhythmias, and cardiomyopathies), spondyloarthropathies (including ankylosing spondylitis, psoriatic arthritis/spondylitis, enteropathic arthritis, reactive arthritis or Reiter's syndrome, and undifferentiated spondyloarthropathies), metabolic disorders (including obesity and diabetes, including type 1 diabetes mellitus, type 2 diabetes mellitus, diabetic neuropathy, peripheral neuropathy, diabetic retinopathy, diabetic ulcerations, retinopathy ulcerations and diabetic macrovasculopathy), anemia, pain (including acute and chronic pains, such as neuropathic pain and post-operative pain, chronic lower back pain, cluster headaches, herpes neuralgia, phantom limb pain, central pain, dental pain, opioid-resistant pain, visceral pain, surgical pain, bone injury pain, pain during labor and delivery, pain resulting from burns, including sunburn, post partum pain, migraine, angina pain, and genitourinary tract-related pain including cystitis), hepatic disorders (including hepatitis, alcoholic hepatitis, viral hepatitis, alcoholic cirrhosis, al antitypsin deficiency, autoimmune cirrhosis, cryptogenic cirrhosis, fulminant hepatitis, hepatitis B and C, and steatohepatitis, cystic fibrosis, primary biliary cirrhosis, sclerosing cholangitis and biliary obstruction), skin and nail disorders (including psoriasis (including chronic plaque psoriasis, guttate psoriasis, inverse psoriasis, pustular psoriasis and other psoriasis disorders), pemphigus vulgaris, scleroderma, atopic dermatitis (eczema), sarcoidosis, erythema nodosum, hidradenitis suppurative, lichen planus, Sweet's syndrome, scleroderma and vitiligo), vasculitides (including Behcet's disease), and other disorders, such as juvenile rheumatoid arthritis (JRA), endometriosis, prostatitis, choroidal neovascularization, sciatica, Sjogren's syndrome, uveitis, wet macular degeneration, osteoporosis, osteoarthritis, active axial spondyloarthritis and non-radiographic axial spondyloarthritis.

As used herein, the term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. In specific embodiments of the invention, the subject is a human.

As used herein, the term “treatment” or “treat” refers to both therapeutic treatment and prophylactic or preventative measures. Those in need of treatment include those already with the disorder, as well as those in which the disorder is to be prevented.

In one embodiment, the invention provides a method of administering a composition comprising a protein with a modulated glycosylation profile, such as an anti-TNFα antibody, or antigen binding fragment thereof, to a subject such that TNFα activity is inhibited or a disorder in which TNFα activity is detrimental is treated. In one embodiment, the TNFα is human TNFα and the subject is a human subject. In one embodiment, the anti-TNFα antibody is adalimumab, also referred to as HUMIRA®.

The compositions comprising a protein with a modulated glycosylation profile can be administered by a variety of methods known in the art. Exemplary routes/modes of administration include subcutaneous injection, intravenous injection or infusion. In certain aspects, a composition comprising a protein with a modulated glycosylation profile may be orally administered. As will be appreciated by the skilled artisan, the route and/or mode of administration will vary depending upon the desired results.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. In certain embodiments it is especially advantageous to formulate parenteral compositions in dosage unit form for ease of administration and uniformity of dosage. Dosage unit form as used herein refers to physically discrete units suited as unitary dosages for the mammalian subjects to be treated; each unit comprising a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a composition comprising a protein with a modulated glycosylation profile of the invention is 0.01-20 mg/kg, or 1-10 mg/kg, or 0.3-1 mg/kg. With respect to a composition comprising a protein such as an anti-TNFα antibody with a modulated glycosylation profile, or antigen-binding portion thereof, such as adalimumab, an exemplary dose is 40 mg every other week. In some embodiments, in particular for treatment of ulcerative colitis or Crohn's disease, an exemplary dose includes an initial dose (Day 1) of 160 mg (e.g., four 40 mg injections in one day or two 40 mg injections per day for two consecutive days), a second dose two weeks later of 80 mg, and a maintenance dose of 40 mg every other week beginning two weeks later. Alternatively, for psoriasis for example, a dosage can include an 80 mg initial dose followed by 40 mg every other week starting one week after the initial dose.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### IV. Pharmaceutical Formulations Containing Compositions Comprising Recombinant Proteins with Modulated Glycosylation Profiles of the Invention

The present invention further provides preparations and formulations comprising compositions comprising a recombinant protein with a modulated glycosylation profile, for example a recombinant protein such as an antibody, antigen-binding portion thereof, or a DVD-Ig, with an increased galactosylation level or amount, an increased or decreased mannosylation level or amount and/or an increased or decreased level or amount of agalactosyl N-glycans. It should be understood that any of the compositions comprising the recombinant proteins with modulated glycosylation profiles, such as antibodies, antibody fragments and DVD-Igs described herein, may be formulated or prepared as described below. In one embodiment, the antibody is an anti-TNFα antibody, or antigen-binding portion thereof.

In certain embodiments, the compositions comprising a protein with a modulated glycosylation profile, of the invention may be formulated with a pharmaceutically acceptable carrier as pharmaceutical (therapeutic) compositions, and may be administered by a variety of methods known in the art. As will be appreciated by the skilled artisan, the route and/or mode of administration will vary depending upon the desired results. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also routinely contain compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the protein with a modulated glycosylation profile (e.g., antibodies or DVD-Igs) of the present invention, and with each other, in a manner such that there is no interaction which would substantially impair the desired pharmaceutical efficacy.

The compositions comprising a protein with a modulated glycosylation profile, of the invention are present in a form known in the art and acceptable for therapeutic uses. In one embodiment, a formulation of the compositions comprising a protein with a modulated glycosylation profile, of the invention is a liquid formulation. In another embodiment, a formulation of the compositions comprising a protein with a modulated glycosylation profile, of the invention is a lyophilized formulation. In a further embodiment, a formulation of the compositions comprising a protein with a modulated glycosylation profile, of the invention is a reconstituted liquid formulation. In one embodiment, a formulation of the compositions comprising a protein with a modulated glycosylation profile, of the invention is a stable liquid formulation. In one embodiment, a liquid formulation of the compositions comprising a protein with a modulated glycosylation profile, of the invention is an aqueous formulation. In another embodiment, the liquid formulation is non-aqueous. In a specific embodiment, a liquid formulation of the compositions comprising a protein with a modulated glycosylation profile, of the invention is an aqueous formulation wherein the aqueous carrier is distilled water.

The formulations of the compositions comprising a protein with a modulated glycosylation profile (e.g., an antibody or a DVD-Ig) in a concentration resulting in a w/v appropriate for a desired dose. The protein with a modulated glycosylation profile may be present in the formulation at a concentration of about 1 mg/ml to about 500 mg/ml, e.g., at a concentration of at least 1 mg/ml, at least 5 mg/ml, at least 10 mg/ml, at least 15 mg/ml, at least 20 mg/ml, at least 25 mg/ml, at least 30 mg/ml, at least 35 mg/ml, at least 40 mg/ml, at least 45 mg/ml, at least 50 mg/ml, at least 55 mg/ml, at least 60 mg/ml, at least 65 mg/ml, at least 70 mg/ml, at least 75 mg/ml, at least 80 mg/ml, at least 85 mg/ml, at least 90 mg/ml, at least 95 mg/ml, at least 100 mg/ml, at least 105 mg/ml, at least 110 mg/ml, at least 115 mg/ml, at least 120 mg/ml, at least 125 mg/ml, at least 130 mg/ml, at least 135 mg/ml, at least 140 mg/ml, at least 150 mg/ml, at least 200 mg/ml, at least 250 mg/ml, or at least 300 mg/ml.

In a specific embodiment, a formulation of compositions comprising a protein with a modulated glycosylation profile, of the invention comprises at least about 100 mg/ml, at least about 125 mg/ml, at least 130 mg/ml, or at least about 150 mg/ml of protein with a modulated glycosylation profile (e.g., an antibody or DVD-Ig) of the invention.

In one embodiment, the concentration of a protein with a modulated glycosylation profile (e.g., antibody or DVD-Ig), which is included in the formulation of the invention, is between about 1 mg/ml and about 25 mg/ml, between about 1 mg/ml and about 200 mg/ml, between about 25 mg/ml and about 200 mg/ml, between about 50 mg/ml and about 200 mg/ml, between about 75 mg/ml and about 200 mg/ml, between about 100 mg/ml and about 200 mg/ml, between about 125 mg/ml and about 200 mg/ml, between about 150 mg/ml and about 200 mg/ml, between about 25 mg/ml and about 150 mg/ml, between about 50 mg/ml and about 150 mg/ml, between about 75 mg/ml and about 150 mg/ml, between about 100 mg/ml and about 150 mg/ml, between about 125 mg/ml and about 150 mg/ml, between about 25 mg/ml and about 125 mg/ml, between about 50 mg/ml and about 125 mg/ml, between about 75 mg/ml and about 125 mg/ml, between about 100 mg/ml and about 125 mg/ml, between about 25 mg/ml and about 100 mg/ml, between about 50 mg/ml and about 100 mg/ml, between about 75 mg/ml and about 100 mg/ml, between about 25 mg/ml and about 75 mg/ml, between about 50 mg/ml and about 75 mg/ml, or between about 25 mg/ml and about 50 mg/ml.

In a specific embodiment, a formulation of the compositions comprising a protein with a modulated glycosylation profile of the invention comprises between about 90 mg/ml and about 110 mg/ml or between about 100 mg/ml and about 210 mg/ml of a protein with a modulated glycosylation profile (e.g., an antibody or DVD-Ig).

The formulations of the compositions comprising a protein with a modulated glycosylation profile of the invention comprising a protein (e.g., an antibody or DVD-Ig) may further comprise one or more active compounds as necessary for the particular indication being treated, typically those with complementary activities that do not adversely affect each other. Such additional active compounds are suitably present in combination in amounts that are effective for the purpose intended.

The formulations of the compositions comprising a protein with a modulated glycosylation profile may be prepared for storage by mixing the protein (e.g., antibody or DVD-Ig) having the desired degree of purity with optional physiologically acceptable carriers, excipients or stabilizers, including, but not limited to buffering agents, saccharides, salts, surfactants, solubilizers, polyols, diluents, binders, stabilizers, salts, lipophilic solvents, amino acids, chelators, preservatives, or the like (Goodman and Gilman's The Pharmacological Basis of Therapeutics, 12^{th} edition, L. Brunton, et al. and Remington's Pharmaceutical Sciences, 16th edition, Osol, A. Ed. (1999)), in the form of lyophilized formulations or aqueous solutions at a desired final concentration. Acceptable carriers, excipients, or stabilizers are nontoxic to recipients at the dosages and concentrations employed, and include buffers such as histidine, phosphate, citrate, glycine, acetate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrolidone; amino acids such as glycine, glutamine, asparagine, histidine, arginine, or lysine; monosaccharides, disaccharides, and other carbohydrates including trehalose, glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, polysorbate 80, PLURONICS™ or polyethylene glycol (PEG).

The buffering agent may be histidine, citrate, phosphate, glycine, or acetate. The saccharide excipient may be trehalose, sucrose, mannitol, maltose or raffinose. The surfactant may be polysorbate 20, polysorbate 40, polysorbate 80, or Pluronic F68. The salt may be NaCl, KCl, MgCl₂, or CaCl₂

The formulations of the compositions comprising a protein with a modulated glycosylation profile of the invention may include a buffering or pH adjusting agent to provide improved pH control. A formulation of the invention may have a pH of between about 3.0 and about 9.0, between about 4.0 and about 8.0, between about 5.0 and about 8.0, between about 5.0 and about 7.0, between about 5.0 and about 6.5, between about 5.5 and about 8.0, between about 5.5 and about 7.0, or between about 5.5 and about 6.5. In a further embodiment, a formulation of the invention has a pH of about 3.0, about 3.5, about 4.0, about 4.5, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.5, about 8.0, about 8.5, or about 9.0. In a specific embodiment, a formulation of the invention has a pH of about 6.0. One of skill in the art understands that the pH of a formulation generally should not be equal to the isoelectric point of the particular a protein (e.g., antibody or DVD-Ig) to be used in the formulation.

Typically, the buffering agent is a salt prepared from an organic or inorganic acid or base. Representative buffering agents include, but are not limited to, organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. In addition, amino acid components can also function in a buffering capacity. Representative amino acid components which may be utilized in the formulations of the invention as buffering agents include, but are not limited to, glycine and histidine. In certain embodiments, the buffering agent is chosen from histidine, citrate, phosphate, glycine, and acetate. In a specific embodiment, the buffering agent is histidine. In another specific embodiment, the buffering agent is citrate. In yet another specific embodiment, the buffering agent is glycine. The purity of the buffering agent should be at least 98%, or at least 99%, or at least 99.5%. As used herein, the term “purity” in the context of histidine and glycine refers to chemical purity of histidine or glycine as understood in the art, e.g., as described in The Merck Index, 13^{th} ed., O'Neil et al. ed. (Merck & Co., 2001).

Buffering agents are typically used at concentrations between about 1 mM and about 200 mM or any range or value therein, depending on the desired ionic strength and the buffering capacity required. The usual concentrations of conventional buffering agents employed in parenteral formulations can be found in: Pharmaceutical Dosage Form: Parenteral Medications, Volume 1, 2^{nd} Edition, Chapter 5, p. 194, De Luca and Boylan, “Formulation of Small Volume Parenterals”, Table 5: Commonly used additives in Parenteral Products. In one embodiment, the buffering agent is at a concentration of about 1 mM, or of about 5 mM, or of about 10 mM, or of about 15 mM, or of about 20 mM, or of about 25 mM, or of about 30 mM, or of about 35 mM, or of about 40 mM, or of about 45 mM, or of about 50 mM, or of about 60 mM, or of about 70 mM, or of about 80 mM, or of about 90 mM, or of about 100 mM. In one embodiment, the buffering agent is at a concentration of 1 mM, or of 5 mM, or of 10 mM, or of 15 mM, or of 20 mM, or of 25 mM, or of 30 mM, or of 35 mM, or of 40 mM, or of 45 mM, or of 50 mM, or of 60 mM, or of 70 mM, or of 80 mM, or of 90 mM, or of 100 mM. In a specific embodiment, the buffering agent is at a concentration of between about 5 mM and about 50 mM. In another specific embodiment, the buffering agent is at a concentration of between 5 mM and 20 mM.

In certain embodiments, the formulation of the compositions comprising a protein with a modulated glycosylation profile of the invention comprises histidine as a buffering agent. In one embodiment the histidine is present in the formulation of the invention at a concentration of at least about 1 mM, at least about 5 mM, at least about 10 mM, at least about 20 mM, at least about 30 mM, at least about 40 mM, at least about 50 mM, at least about 75 mM, at least about 100 mM, at least about 150 mM, or at least about 200 mM histidine. In another embodiment, a formulation of the invention comprises between about 1 mM and about 200 mM, between about 1 mM and about 150 mM, between about 1 mM and about 100 mM, between about 1 mM and about 75 mM, between about 10 mM and about 200 mM, between about 10 mM and about 150 mM, between about 10 mM and about 100 mM, between about 10 mM and about 75 mM, between about 10 mM and about 50 mM, between about 10 mM and about 40 mM, between about 10 mM and about 30 mM, between about 20 mM and about 75 mM, between about 20 mM and about 50 mM, between about 20 mM and about 40 mM, or between about 20 mM and about 30 mM histidine. In a further embodiment, the formulation comprises about 1 mM, about 5 mM, about 10 mM, about 20 mM, about 25 mM, about 30 mM, about 35 mM, about 40 mM, about 45 mM, about 50 mM, about 60 mM, about 70 mM, about 80 mM, about 90 mM, about 100 mM, about 150 mM, or about 200 mM histidine. In a specific embodiment, a formulation may comprise about 10 mM, about 25 mM, or no histidine.

The formulations of the compositions comprising a protein with a modulated glycosylation profile of the invention may comprise a carbohydrate excipient. Carbohydrate excipients can act, e.g., as viscosity enhancing agents, stabilizers, bulking agents, solubilizing agents, and/or the like. Carbohydrate excipients are generally present at between about 1% to about 99% by weight or volume, e.g., between about 0.1% to about 20%, between about 0.1% to about 15%, between about 0.1% to about 5%, between about 1% to about 20%, between about 5% to about 15%, between about 8% to about 10%, between about 10% and about 15%, between about 15% and about 20%, between 0.1% to 20%, between 5% to 15%, between 8% to 10%, between 10% and 15%, between 15% and 20%, between about 0.1% to about 5%, between about 5% to about 10%, or between about 15% to about 20%. In still other specific embodiments, the carbohydrate excipient is present at 1%, or at 1.5%, or at 2%, or at 2.5%, or at 3%, or at 4%, or at 5%, or at 10%, or at 15%, or at 20%.

Carbohydrate excipients suitable for use in the formulations of the invention include, but are not limited to, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and the like. In one embodiment, the carbohydrate excipients for use in the present invention are chosen from, sucrose, trehalose, lactose, mannitol, and raffinose. In a specific embodiment, the carbohydrate excipient is trehalose. In another specific embodiment, the carbohydrate excipient is mannitol. In yet another specific embodiment, the carbohydrate excipient is sucrose. In still another specific embodiment, the carbohydrate excipient is raffinose. The purity of the carbohydrate excipient should be at least 98%, or at least 99%, or at least 99.5%.

In a specific embodiment, the formulations of the compositions comprising a protein with a modulated glycosylation profile of the invention may comprise trehalose. In one embodiment, a formulation of the invention comprises at least about 1%, at least about 2%, at least about 4%, at least about 8%, at least about 20%, at least about 30%, or at least about 40% trehalose. In another embodiment, a formulation of the invention comprises between about 1% and about 40%, between about 1% and about 30%, between about 1% and about 20%, between about 2% and about 40%, between about 2% and about 30%, between about 2% and about 20%, between about 4% and about 40%, between about 4% and about 30%, or between about 4% and about 20% trehalose. In a further embodiment, a formulation of the invention comprises about 1%, about 2%, about 4%, about 6%, about 8%, about 15%, about 20%, about 30%, or about 40% trehalose. In a specific embodiment, a formulation of the invention comprises about 4%, about 6% or about 15% trehalose.

In certain embodiments, a formulation of the compositions comprising a protein with a modulated glycosylation profile of the invention comprises an excipient. In a specific embodiment, a formulation of the invention comprises at least one excipient chosen from: sugar, salt, surfactant, amino acid, polyol, chelating agent, emulsifier and preservative. In one embodiment, a formulation of the invention comprises a salt, e.g., a salt selected from: NaCl, KCl, CaCl₂, and MgCl₂. In a specific embodiment, the formulation comprises NaCl.

A formulation of the compositions comprising a protein with a modulated glycosylation profile of the invention may comprise at least about 10 mM, at least about 25 mM, at least about 50 mM, at least about 75 mM, at least about 80 mM, at least about 100 mM, at least about 125 mM, at least about 150 mM, at least about 175 mM, at least about 200 mM, or at least about 300 mM sodium chloride (NaCl). In a further embodiment, the formulation may comprise between about 10 mM and about 300 mM, between about 10 mM and about 200 mM, between about 10 mM and about 175 mM, between about 10 mM and about 150 mM, between about 25 mM and about 300 mM, between about 25 mM and about 200 mM, between about 25 mM and about 175 mM, between about 25 mM and about 150 mM, between about 50 mM and about 300 mM, between about 50 mM and about 200 mM, between about 50 mM and about 175 mM, between about 50 mM and about 150 mM, between about 75 mM and about 300 mM, between about 75 mM and about 200 mM, between about 75 mM and about 175 mM, between about 75 mM and about 150 mM, between about 100 mM and about 300 mM, between about 100 mM and about 200 mM, between about 100 mM and about 175 mM, or between about 100 mM and about 150 mM sodium chloride. In a further embodiment, the formulation may comprise about 10 mM, about 25 mM, about 50 mM, about 75 mM, about 80 mM, about 100 mM, about 125 mM, about 150 mM, about 175 mM, about 200 mM, or about 300 mM sodium chloride.

A formulation of the compositions comprising a protein with a modulated glycosylation profile of the invention may also comprise an amino acid, e.g., lysine, arginine, glycine, histidine or an amino acid salt. The formulation may comprise at least about 1 mM, at least about 10 mM, at least about 25 mM, at least about 50 mM, at least about 100 mM, at least about 150 mM, at least about 200 mM, at least about 250 mM, at least about 300 mM, at least about 350 mM, or at least about 400 mM of an amino acid. In another embodiment, the formulation may comprise between about 1 mM and about 100 mM, between about 10 mM and about 150 mM, between about 25 mM and about 250 mM, between about 25 mM and about 300 mM, between about 25 mM and about 350 mM, between about 25 mM and about 400 mM, between about 50 mM and about 250 mM, between about 50 mM and about 300 mM, between about 50 mM and about 350 mM, between about 50 mM and about 400 mM, between about 100 mM and about 250 mM, between about 100 mM and about 300 mM, between about 100 mM and about 400 mM, between about 150 mM and about 250 mM, between about 150 mM and about 300 mM, or between about 150 mM and about 400 mM of an amino acid. In a further embodiment, a formulation of the invention comprises about 1 mM, 1.6 mM, 25 mM, about 50 mM, about 100 mM, about 150 mM, about 200 mM, about 250 mM, about 300 mM, about 350 mM, or about 400 mM of an amino acid.

The formulations of the compositions comprising a protein with a modulated glycosylation profile of the invention may further comprise a surfactant. The term “surfactant” as used herein refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials. Pharmaceutically acceptable surfactants like polysorbates (e.g., polysorbates 20 or 80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™ PF68, etc.), can optionally be added to the formulations of the invention to reduce aggregation. In one embodiment, a formulation of the invention comprises Polysorbate 20, Polysorbate 40, Polysorbate 60, or Polysorbate 80. Surfactants are particularly useful if a pump or plastic container is used to administer the formulation. The presence of a pharmaceutically acceptable surfactant mitigates the propensity for the protein to aggregate. The formulations may comprise a polysorbate which is at a concentration ranging from between about 0.001% to about 1%, or about 0.001% to about 0.1%, or about 0.01% to about 0.1%. In other specific embodiments, the formulations of the invention comprise a polysorbate which is at a concentration of 0.001%, or 0.002%, or 0.003%, or 0.004%, or 0.005%, or 0.006%, or 0.007%, or 0.008%, or 0.009%, or 0.01%, or 0.015%, or 0.02%.

The formulations of the compositions comprising a protein with a modulated glycosylation profile of the invention may optionally further comprise other common excipients and/or additives including, but not limited to, diluents, binders, stabilizers, lipophilic solvents, preservatives, adjuvants, or the like. Pharmaceutically acceptable excipients and/or additives may be used in the formulations of the invention. Commonly used excipients/additives, such as pharmaceutically acceptable chelators (for example, but not limited to, EDTA, DTPA or EGTA) can optionally be added to the formulations of the invention to reduce aggregation. These additives are particularly useful if a pump or plastic container is used to administer the formulation.

Preservatives, such as phenol, m-cresol, p-cresol, o-cresol, chlorocresol, benzyl alcohol, phenylmercuric nitrite, phenoxyethanol, formaldehyde, chlorobutanol, magnesium chloride (for example, but not limited to, hexahydrate), alkylparaben (methyl, ethyl, propyl, butyl and the like), benzalkonium chloride, benzethonium chloride, sodium dehydroacetate and thimerosal, or mixtures thereof can optionally be added to the formulations of the invention at any suitable concentration such as between about 0.001% to about 5%, or any range or value therein. The concentration of preservative used in the formulations of the invention is a concentration sufficient to yield a microbial effect. Such concentrations are dependent on the preservative selected and are readily determined by the skilled artisan.

Other contemplated excipients/additives, which may be utilized in the formulations of the invention include, for example, flavoring agents, antimicrobial agents, sweeteners, antioxidants, antistatic agents, lipids such as phospholipids or fatty acids, steroids such as cholesterol, protein excipients such as serum albumin (human serum albumin (HSA), recombinant human albumin (rHA), gelatin, casein, salt-forming counterions such as sodium and the like. These and additional known pharmaceutical excipients and/or additives suitable for use in the formulations of the invention are known in the art, e.g., as listed in “Remington: The Science & Practice of Pharmacy”, 21^{st} ed., Lippincott Williams & Wilkins, (2005), and in the “Physician's Desk Reference”, 60^{th} ed., Medical Economics, Montvale, N.J. (2005). Pharmaceutically acceptable carriers can be routinely selected that are suitable for the mode of administration, solubility and/or stability of protein with a modulated glycosylation profile (e.g., an antibody or DVD-Ig), as well known those in the art or as described herein.

In one embodiment, the compositions comprising a protein with a modulated glycosylation profile of the invention are formulated with the same or similar excipients and buffers as are present in the commercial adalimumab (HUMIRA®) formulation, as described in the “Highlights of Prescribing Information” for HUMIRA® (adalimumab) Injection (Revised January 2008) the contents of which are hereby incorporated herein by reference. For example, each prefilled syringe of HUMIRA®, which is administered subcutaneously, delivers 0.8 mL (40 mg) of drug product to the subject. Each 0.8 mL of HUMIRA® contains 40 mg adalimumab, 4.93 mg sodium chloride, 0.69 mg monobasic sodium phosphate dihydrate, 1.22 mg dibasic sodium phosphate dihydrate, 0.24 mg sodium citrate, 1.04 mg citric acid monohydrate, 9.6 mg mannitol, 0.8 mg polysorbate 80, and water for Injection, USP. Sodium hydroxide is added as necessary to adjust pH.

It will be understood by one skilled in the art that the formulations of the compositions comprising a protein with a modulated glycosylation profile of the invention may be isotonic with human blood, wherein the formulations of the invention have essentially the same osmotic pressure as human blood. Such isotonic formulations will generally have an osmotic pressure from about 250 mOSm to about 350 mOSm. Isotonicity can be measured by, for example, using a vapor pressure or ice-freezing type osmometer. Tonicity of a formulation is adjusted by the use of tonicity modifiers. “Tonicity modifiers” are those pharmaceutically acceptable inert substances that can be added to the formulation to provide an isotonity of the formulation. Tonicity modifiers suitable for this invention include, but are not limited to, saccharides, salts and amino acids.

In certain embodiments, the formulations of the compositions comprising a protein with a modulated glycosylation profile of the invention have an osmotic pressure from about 100 mOSm to about 1200 mOSm, or from about 200 mOSm to about 1000 mOSm, or from about 200 mOSm to about 800 mOSm, or from about 200 mOSm to about 600 mOSm, or from about 250 mOSm to about 500 mOSm, or from about 250 mOSm to about 400 mOSm, or from about 250 mOSm to about 350 mOSm.

The concentration of any one component or any combination of various components, of the formulations of the compositions comprising a protein with a modulated glycosylation profile of the invention is adjusted to achieve the desired tonicity of the final formulation. For example, the ratio of the carbohydrate excipient to protein with a modulated glycosylation profile (e.g., antibody or DVD-Ig) may be adjusted according to methods known in the art (e.g., U.S. Pat. No. 6,685,940). In certain embodiments, the molar ratio of the carbohydrate excipient to protein with a modulated glycosylation profile (e.g., antibody or DVD-Ig) may be from about 100 moles to about 1000 moles of carbohydrate excipient to about 1 mole of protein with a modulated glycosylation profile, or from about 200 moles to about 6000 moles of carbohydrate excipient to about 1 mole of protein with a modulated glycosylation profile, or from about 100 moles to about 510 moles of carbohydrate excipient to about 1 mole of protein with a modulated glycosylation profile, or from about 100 moles to about 600 moles of carbohydrate excipient to about 1 mole of protein with a modulated glycosylation profile.

The desired isotonicity of the final formulation may also be achieved by adjusting the salt concentration of the formulations. Pharmaceutically acceptable salts and those suitable for this invention as tonicity modifiers include, but are not limited to, sodium chloride, sodium succinate, sodium sulfate, potassium chloride, magnesium chloride, magnesium sulfate, and calcium chloride. In specific embodiments, formulations of the invention comprise NaCl, MgCl₂, and/or CaCl₂. In one embodiment, concentration of NaCl is between about 75 mM and about 150 mM. In another embodiment, concentration of MgCl₂ is between about 1 mM and about 100 mM. Pharmaceutically acceptable amino acids including those suitable for this invention as tonicity modifiers include, but are not limited to, proline, alanine, L-arginine, asparagine, L-aspartic acid, glycine, serine, lysine, and histidine.

In one embodiment the formulations of the compositions comprising a protein with a modulated glycosylation profile of the invention are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with proteins of interest (e.g., antibodies), even trace amounts of harmful and dangerous endotoxin must be removed. In certain specific embodiments, the endotoxin and pyrogen levels in the composition are less than 10 EU/mg, or less than 5 EU/mg, or less than 1 EU/mg, or less than 0.1 EU/mg, or less than 0.01 EU/mg, or less than 0.001 EU/mg.

When used for in vivo administration, the formulations of the compositions comprising a protein with a modulated glycosylation profile of the invention should be sterile. The formulations of the invention may be sterilized by various sterilization methods, including sterile filtration, radiation, etc. In one embodiment, the protein with a modulated glycosylation profile (e.g., antibody or DVD-Ig) formulation is filter-sterilized with a presterilized 0.22-micron filter. Sterile compositions for injection can be formulated according to conventional pharmaceutical practice as described in “Remington: The Science & Practice of Pharmacy”, 21^{st} ed., Lippincott Williams & Wilkins, (2005). Formulations comprising proteins of interest (e.g., antibody or DVD-Ig.), such as those disclosed herein, ordinarily will be stored in lyophilized form or in solution. It is contemplated that sterile compositions comprising proteins of interest (e.g., antibody or DVD-Ig) are placed into a container having a sterile access port, for example, an intravenous solution bag or vial having an adapter that allows retrieval of the formulation, such as a stopper pierceable by a hypodermic injection needle. In one embodiment, a composition of the invention is provided as a pre-filled syringe.

In one embodiment, a formulation of the compositions comprising a protein with a modulated glycosylation profile of the invention is a lyophilized formulation. The term “lyophilized” or “freeze-dried” includes a state of a substance that has been subjected to a drying procedure such as lyophilization, where at least 50% of moisture has been removed.

The phrase “bulking agent” includes a compound that is pharmaceutically acceptable and that adds bulk to a lyo cake. Bulking agents known to the art include, for example, carbohydrates, including simple sugars such as dextrose, ribose, fructose and the like, alcohol sugars such as mannitol, inositol and sorbitol, disaccharides including trehalose, sucrose and lactose, naturally occurring polymers such as starch, dextrans, chitosan, hyaluronate, proteins (e.g., gelatin and serum albumin), glycogen, and synthetic monomers and polymers.

A “lyoprotectant” is a molecule which, when combined with a protein with a modulated glycosylation profile (such as an antibody or DVD-Ig of the invention), significantly prevents or reduces chemical and/or physical instability of the protein upon lyophilization and subsequent storage. Lyoprotectants include, but are not limited to, sugars and their corresponding sugar alcohols; an amino acid such as monosodium glutamate or histidine; a methylamine such as betaine; a lyotropic salt such as magnesium sulfate; a polyol such as trihydric or higher molecular weight sugar alcohols, e.g., glycerin, dextran, erythritol, glycerol, arabitol, xylitol, sorbitol, and mannitol; propylene glycol; polyethylene glycol; PLURONICS™; and combinations thereof. Additional examples of lyoprotectants include, but are not limited to, glycerin and gelatin, and the sugars mellibiose, melezitose, raffinose, mannotriose and stachyose. Examples of reducing sugars include, but are not limited to, glucose, maltose, lactose, maltulose, iso-maltulose and lactulose. Examples of non-reducing sugars include, but are not limited to, non-reducing glycosides of polyhydroxy compounds selected from sugar alcohols and other straight chain polyalcohols. Examples of sugar alcohols include, but are not limited to, monoglycosides, compounds obtained by reduction of disaccharides such as lactose, maltose, lactulose and maltulose. The glycosidic side group can be either glucosidic or galactosidic. Additional examples of sugar alcohols include, but are not limited to, glucitol, maltitol, lactitol and iso-maltulose. In specific embodiments, trehalose or sucrose is used as a lyoprotectant.

The lyoprotectant is added to the pre-lyophilized formulation in a “lyoprotecting amount” which means that, following lyophilization of the protein in the presence of the lyoprotecting amount of the lyoprotectant, the protein essentially retains its physical and chemical stability and integrity upon lyophilization and storage.

In one embodiment, the molar ratio of a lyoprotectant (e.g., trehalose) and protein with a modulated glycosylation profile (e.g., antibody or DVD-Ig) molecules of a formulation of the invention is at least about 10, at least about 50, at least about 100, at least about 200, or at least about 300. In another embodiment, the molar ratio of a lyoprotectant (e.g., trehalose) and protein with a modulated glycosylation profile molecules of a formulation of the invention is about 1, is about 2, is about 5, is about 10, about 50, about 100, about 200, or about 300.

A “reconstituted” formulation is one which has been prepared by dissolving a lyophilized protein with a modulated glycosylation profile (e.g., antibody or DVD-Ig) formulation in a diluent such that the protein with a modulated glycosylation profile is dispersed in the reconstituted formulation. The reconstituted formulation is suitable for administration (e.g., parenteral administration) to a patient to be treated with the protein with a modulated glycosylation profile and, in certain embodiments of the invention, may be one which is suitable for intravenous administration.

The “diluent” of interest herein is one which is pharmaceutically acceptable (safe and non-toxic for administration to a human) and is useful for the preparation of a liquid formulation, such as a formulation reconstituted after lyophilization. In some embodiments, diluents include, but are not limited to, sterile water, bacteriostatic water for injection (BWFI), a pH buffered solution (e.g., phosphate-buffered saline), sterile saline solution, Ringer's solution or dextrose solution. In an alternative embodiment, diluents can include aqueous solutions of salts and/or buffers.

In certain embodiments, a formulation of the compositions comprising a protein with a modulated glycosylation profile of the invention is a lyophilized formulation comprising a protein with a modulated glycosylation profile (e.g., antibody or DVD-Ig) of the invention, wherein at least about 90%, at least about 95%, at least about 97%, at least about 98%, or at least about 99% of the protein with a modulated glycosylation profile may be recovered from a vial upon shaking the vial for 4 hours at a speed of 400 shakes per minute wherein the vial is filled to half of its volume with the formulation. In another embodiment, a formulation of the invention is a lyophilized formulation comprising a protein with a modulated glycosylation profile of the invention, wherein at least about 90%, at least about 95%, at least about 97%, at least about 98%, or at least about 99% of the protein with a modulated glycosylation profile may be recovered from a vial upon subjecting the formulation to three freeze/thaw cycles wherein the vial is filled to half of its volume with the formulation. In a further embodiment, a formulation of the invention is a lyophilized formulation comprising a protein with a modulated glycosylation profile of the invention, wherein at least about 90%, at least about 95%, at least about 97%, at least about 98%, or at least about 99% of the protein with a modulated glycosylation profile may be recovered by reconstituting a lyophilized cake generated from the formulation.

In one embodiment, a reconstituted liquid formulation may comprise a protein with a modulated glycosylation profile (e.g., antibody or DVD-Ig) of the invention at the same concentration as the pre-lyophilized liquid formulation.

In another embodiment, a reconstituted liquid formulation may comprise a protein with a modulated glycosylation profile (e.g., antibody or DVD-Ig) of the invention at a higher concentration than the pre-lyophilized liquid formulation, e.g., about 2 fold, about 3 fold, about 4 fold, about 5 fold, about 6 fold, about 7 fold, about 8 fold, about 9 fold, or about 10 fold higher concentration of a protein with a modulated glycosylation profile than the pre-lyophilized liquid formulation.

In yet another embodiment, a reconstituted liquid formulation may comprise a protein with a modulated glycosylation profile (e.g., antibody or DVD-Ig) of the invention at a lower concentration than the pre-lyophilized liquid formulation, e.g., about 2 fold, about 3 fold, about 4 fold, about 5 fold, about 6 fold, about 7 fold, about 8 fold, about 9 fold or about 10 fold lower concentration of a protein with a modulated glycosylation profile than the pre-lyophilized liquid formulation.

The pharmaceutical formulations of the compositions comprising a protein with a modulated glycosylation profile, of the invention are typically stable formulations, e.g., stable at room temperature.

The terms “stability” and “stable” as used herein in the context of a formulation comprising a protein with a modulated glycosylation profile (e.g., an antibody or DVD-Ig) of the invention refer to the resistance of the protein in the formulation to aggregation, degradation or fragmentation under given manufacture, preparation, transportation and storage conditions. The “stable” formulations of the invention retain biological activity under given manufacture, preparation, transportation and storage conditions. The stability of the protein with a modulated glycosylation profile can be assessed by degrees of aggregation, degradation or fragmentation, as measured by HPSEC, static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference formulation. For example, a reference formulation may be a reference standard frozen at −70° C. consisting of 10 mg/ml of a protein with a modulated glycosylation profile of the invention in PBS.

Therapeutic formulations of the compositions comprising a protein with a modulated glycosylation profile of the invention may be formulated for a particular dosage. Dosage regimens may be adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the protein with a modulated glycosylation profile (e.g., antibody or DVD-Ig) of the invention, and the particular therapeutic effect to be achieved, and (b) the limitations inherent in the art of compounding such a protein with a modulated glycosylation profile for the treatment of sensitivity in individuals.

Therapeutic compositions of the compositions comprising a protein with a modulated glycosylation profile of the invention, can be formulated for particular routes of administration, such as oral, nasal, pulmonary, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. By way of example, in certain embodiments, the proteins with modulated glycosylation profiles (including fragments of the protein with a modulated glycosylation profile) are formulated for intravenous administration. In certain other embodiments, the proteins with modulated glycosylation profiles (e.g., antibody or DVD-Ig), of the invention, including fragments of the proteins with modulated glycosylation profiles (e.g., antibody fragments) of the invention, are formulated for local delivery to the cardiovascular system, for example, via catheter, stent, wire, intramyocardial delivery, intrapericardial delivery, or intraendocardial delivery.

Formulations of the compositions comprising a protein with a modulated glycosylation profile of the invention, which are suitable for topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required (U.S. Pat. Nos. 7,378,110; 7,258,873; 7,135,180; 7,923,029; and US Publication No. 20040042972).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Actual dosage levels of the active ingredients in the pharmaceutical compositions of the compositions comprising a protein with a modulated glycosylation profile of the invention, may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

In certain embodiments, the proteins with modulated glycosylation profiles (e.g., antibody or DVD-Ig) of the invention can be formulated to ensure proper distribution in vivo. For example, the blood-brain barrier (BBB) excludes many highly hydrophilic compounds. To ensure that the therapeutic compounds of the invention can cross the BBB (if desired), they can be formulated, for example, in liposomes. For methods of manufacturing liposomes, see, e.g., U.S. Pat. Nos. 4,522,811; 5,374,548; 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant Protein A receptor (Briscoe et al. (1995) Am. J. Physiol. 1233:134), different species of which may comprise the formulations of the invention, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273. In one embodiment of the invention, the therapeutic compounds of the invention are formulated in liposomes; in another embodiment, the liposomes include a targeting moiety. In another embodiment, the therapeutic compounds in the liposomes are delivered by bolus injection to a site proximal to the desired area. When administered in this manner, the composition must be fluid to the extent that easy syringability exists. It must be stable under the conditions of manufacture and storage and may be preserved against the contaminating action of microorganisms such as bacteria and fungi. Additionally or alternatively, the proteins with modulated glycosylation profiles (e.g., antibodies or DVD-Igs) of the invention may be delivered locally to the brain to mitigate the risk that the blood brain barrier slows effective delivery.

In certain embodiments, the compositions comprising a protein with a modulated glycosylation profile of the invention may be administered with medical devices known in the art. For example, in certain embodiments a protein with a modulated glycosylation profile (e.g., antibody or DVD-Ig) or a fragment of protein with a modulated glycosylation profile (e.g., antibody fragment) is administered locally via a catheter, stent, wire, or the like. For example, in one embodiment, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

The efficient dosages and the dosage regimens for the compositions comprising a protein with a modulated glycosylation profile of the invention depend on the disease or condition to be treated and can be determined by the persons skilled in the art. One of ordinary skill in the art would be able to determine such amounts based on such factors as the subject's size, the severity of the subject's symptoms, and the particular composition or route of administration selected.

### VI. Kits and Articles of Manufacture Comprising the Compositions Comprising Recombinant Proteins with Modulated Glycosylation Profiles of the Invention

Also within the scope of the present invention are kits comprising the compositions comprising a recombinant protein with a modulated glycosylation profile, for example a recombinant protein such as adalimumab, with a modulated, e.g., increased or decreased, mannosylation level and instructions for use. The term “kit” as used herein refers to a packaged product comprising components with which to administer the recombinant protein with a modulated glycosylation profile (e.g., antibody, or antigen-binding portion thereof, or DVD-Ig), of the invention for treatment of a disease or disorder. The kit may comprise a box or container that holds the components of the kit. The box or container is affixed with a label or a Food and Drug Administration approved protocol. The box or container holds components of the invention which may be contained within plastic, polyethylene, polypropylene, ethylene, or propylene vessels. The vessels can be capped-tubes or bottles. The kit can also include instructions for administering a recombinant protein with a modulated glycosylation profile (e.g., an antibody or a DVD-Ig) of the invention.

The kit can further contain one more additional reagents, such as an immunosuppressive reagent, a cytotoxic agent or a radiotoxic agent or one or more additional proteins of interest of the invention (e.g., an antibody having a complementary activity which binds to an epitope in the TNFα antigen distinct from a first anti-TNFα antibody). Kits typically include a label indicating the intended use of the contents of the kit. The term label includes any writing, or recorded material supplied on or with the kit, or which otherwise accompanies the kit.

The invention also provides a pharmaceutical pack or kit comprising one or more containers filled with a liquid formulation or lyophilized formulation of a protein with a modulated glycosylation profile (e.g., an antibody, or antibody fragment thereof, or a DVD-Ig) of the invention. In one embodiment, a container filled with a liquid formulation of the invention is a pre-filled syringe. In a specific embodiment, the formulations of the invention are formulated in single dose vials as a sterile liquid. For example, the formulations may be supplied in 3 cc USP Type I borosilicate amber vials (West Pharmaceutical Services—Part No. 6800-0675) with a target volume of 1.2 mL. Optionally associated with such container(s) can be a notice in the form prescribed by a governmental agency regulating the manufacture, use or sale of pharmaceuticals or biological products, which notice reflects approval by the agency of manufacture, use or sale for human administration.

In one embodiment, a container filled with a liquid formulation of the invention is a pre-filled syringe. Any pre-filled syringe known to one of skill in the art may be used in combination with a liquid formulation of the invention. Pre-filled syringes that may be used are described in, for example, but not limited to, PCT Publications WO05032627, WO08094984, WO9945985, WO03077976, U.S. Pat. No. 6,792,743, U.S. Pat. No. 5,607,400, U.S. Pat. No. 5,893,842, U.S. Pat. No. 7,081,107, U.S. Pat. No. 7,041,087, U.S. Pat. No. 5,989,227, U.S. Pat. No. 6,807,797, U.S. Pat. No. 6,142,976, U.S. Pat. No. 5,899,889, U.S. Pat. No. 7,699,811, U.S. Pat. No. 7,540,382, U.S. Pat. No. 7,998,120, U.S. Pat. No. 7,645,267, and US Patent Publication No. US20050075611. Pre-filled syringes may be made of various materials. In one embodiment a pre-filled syringe is a glass syringe. In another embodiment a pre-filled syringe is a plastic syringe. One of skill in the art understands that the nature and/or quality of the materials used for manufacturing the syringe may influence the stability of a protein formulation stored in the syringe. For example, it is understood that silicon based lubricants deposited on the inside surface of the syringe chamber may affect particle formation in the protein formulation. In one embodiment, a pre-filled syringe comprises a silicone based lubricant. In one embodiment, a pre-filled syringe comprises baked on silicone. In another embodiment, a pre-filled syringe is free from silicone based lubricants. One of skill in the art also understands that small amounts of contaminating elements leaching into the formulation from the syringe barrel, syringe tip cap, plunger or stopper may also influence stability of the formulation. For example, it is understood that tungsten introduced during the manufacturing process may adversely affect formulation stability. In one embodiment, a pre-filled syringe may comprise tungsten at a level above 500 ppb. In another embodiment, a pre-filled syringe is a low tungsten syringe. In another embodiment, a pre-filled syringe may comprise tungsten at a level between about 500 ppb and about 10 ppb, between about 400 ppb and about 10 ppb, between about 300 ppb and about 10 ppb, between about 200 ppb and about 10 ppb, between about 100 ppb and about 10 ppb, between about 50 ppb and about 10 ppb, between about 25 ppb and about 10 ppb.

In certain embodiments, kits comprising a recombinant protein with a modulated glycosylation profile such as an increased galactosylation level or amount, an increased or decreased mannosylation level or amount and/or an increased or decreased level or amount of agalactosyl N-glycans (e.g., an antibody or DVD-Ig) of the invention are also provided that are useful for various purposes, e.g., research and diagnostic including for purification or immunoprecipitation of a recombinant protein with a modulated glycosylation profile from cells, detection of the recombinant protein with a modulated glycosylation profile in vitro or in vivo. For isolation and purification of a protein with a modulated glycosylation profile, the kit may contain an antibody coupled to beads (e.g., sepharose beads). Kits may be provided which contain the antibodies for detection and quantitation of a protein with a modulated glycosylation profile in vitro, e.g., in an ELISA or a Western blot. As with the article of manufacture, the kit comprises a container and a label or package insert on or associated with the container. The container holds a composition comprising at least one protein with a modulated glycosylation profile (e.g., antibody or DVD-Ig) of the invention. Additional containers may be included that contain, e.g., diluents and buffers, control proteins with modulated glycosylation profiles (e.g., antibody or DVD-Ig). The label or package insert may provide a description of the composition as well as instructions for the intended in vitro or diagnostic use.

The present invention also encompasses a finished packaged and labeled pharmaceutical product. This article of manufacture includes the appropriate unit dosage form in an appropriate vessel or container such as a glass vial, pre-filled syringe or other container that is hermetically sealed. In one embodiment, the unit dosage form is provided as a sterile particulate free solution comprising a protein with a modulated glycosylation profile (e.g., an antibody or DVD-Ig) that is suitable for parenteral administration. In another embodiment, the unit dosage form is provided as a sterile lyophilized powder comprising a protein with a modulated glycosylation profile (e.g., an antibody or DVD-Ig) that is suitable for reconstitution.

In one embodiment, the unit dosage form is suitable for intravenous, intramuscular, intranasal, oral, topical or subcutaneous delivery. Thus, the invention encompasses sterile solutions suitable for each delivery route. The invention further encompasses sterile lyophilized powders that are suitable for reconstitution.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question, as well as how and how frequently to administer the pharmaceutical. In other words, the article of manufacture includes instruction means indicating or suggesting a dosing regimen including, but not limited to, actual doses, monitoring procedures, and other monitoring information.

Specifically, the invention provides an article of manufacture comprising packaging material, such as a box, bottle, tube, vial, container, pre-filled syringe, sprayer, insufflator, intravenous (i.v.) bag, envelope and the like; and at least one unit dosage form of a pharmaceutical agent contained within the packaging material, wherein the pharmaceutical agent comprises a liquid formulation containing a protein with a modulated glycosylation profile (e.g., an antibody or DVD-Ig). The packaging material includes instruction means which indicate how that the protein with a modulated glycosylation profile (e.g., antibody or DVD-Ig) can be used to prevent, treat and/or manage one or more symptoms associated with a disease or disorder.

The present invention is further illustrated by the following examples which should not be construed as limiting.

### Examples

### Materials & Methods of the Examples

Cell Culture Media

Media 1, which is a chemically defined growth or production medium, was prepared from basal IVGN CD media (proprietary formulation). For preparation of the IVGN CD media formulation, the media was supplemented with L-glutamine, sodium bicarbonate, sodium chloride, and a methotrexate solution. The production media consisted of all the same components found in the growth medium, however, the production medium excludes methotrexate. For cell line 1 and mAb2, the medium was also supplemented with insulin. In addition, 10 mM or 5 mM of galactose (Sigma, G5388) and 0.2 μM or 10 μM of manganese (Sigma, M1787) were supplemented into the production medium for cell line 3 and cell line 1, respectively. The osmolality of the media was adjusted via modulation of sodium chloride concentration. All media were filtered using a filter system (0.22 μm PES) and stored at 4° C. until usage.

Media 2 is a hydrolysate-based media, which contains basal proprietary media, Bacto TC Yeastolate and Phytone Peptone.

Production Cultures

Production cultures were initiated in 3 L Bioreactors (Applikon). The bioreactors (1.5-2.0 L working volume) were run at the following conditions (except for the different experimental conditions): 35° C., 30% DO (dissolved oxygen), 200 rpm, pH profile from 7.1 to 6.9 in three days and pH 6.9 thereafter. In all experiments, the cells were transferred from the wavebag to the production stage at a split ratio of 1:5.6 (except mAb2 with a ratio of 1:5). When the media glucose concentration decreased to a concentration less than 3 g/L, approximately 1.25% (v/v) of 40% glucose stock solution was fed.

The harvest procedure of reactors involved the centrifugation of the culture sample at 3,000 RPM for 30 minutes and storage of the supernatant in PETG bottles at −80° C. before submission for protein A purification and WCX-10 analysis.

Cell Source and Adaptation Cultures

Two adalimumab producing CHO cell lines (i.e., cell line 1 and cell line 3) and a mAB2 producing cell line were employed. Upon thawing, adalimumab producing cell line 3 was cultured in chemically defined growth media (media 1) in a combination of vented shake flasks on a shaker platform at 140 rpm and 20 L wave bags. Cultures were propagated in a 36° C., 5% CO₂ incubator to obtain the required number of cells to be able to initiate production stage cultures.

Upon thawing, adalimumab producing cell line 1 was cultured in a hydrolysate based growth media (media 2) in a combination of vented shake flasks on a shaker platform at 110 rpm and 20 L wave bags. Cultures were propagated in a 35° C., 5% CO₂ incubator. In some cases, the culture might be transferred into a seed reactor with pH 7.1, 35° C. and 30% DO. The culture would be adapted to either media 1 or media 2 by propagation in a 10 L or 20 L wavebag for 7 to 13 days with one or two passages before initiating production stage cultures.

Upon thawing, mAb2 producing cells were cultured in media 1 in a combination of vented non-baffled shake flasks (Corning) on a shaker platform at 140 RPM and 20 L wave bags (GE). Cultures were propagated in a 35° C., 5% CO₂ incubator to obtain the required number of cells to be able to initiate production stage cultures.

Oligosaccharide Assay

The oligosaccharides are released from the protein by enzymatic digestion with N-glycanase. Once the glycans are released, the free reducing end of each glycan is labeled by reductive amination with a fluorescent tag, 2-aminobenzamide (2-AB). The resulting labeled glycans are separated by normal-phase HPLC (NP-HPLC) in acetonitrile: 50 mM ammonium formate, pH 4.4, and detected using a fluorescence detector. Quantitation is based on the relative area percent of detected sugars. The relative area percents of the agalactosyl fucosylated biantennary oligosaccharides (NGA2F+[NGA2F-GlcNAc]), the galactose-containing fucosylated biantennary oligosaccharides (NA1F+NA2F), the oligomannoses (M5+M6) and the proportion of NA1F form as a percent of total galactose-containing fucosylated biantennary oligosaccharides in the sample(s) are reported.

### Example 1: The Effect of Dissolved Oxygen on the Level of Oligomannose for Cell Line 1 in Media 2 at 35° C.

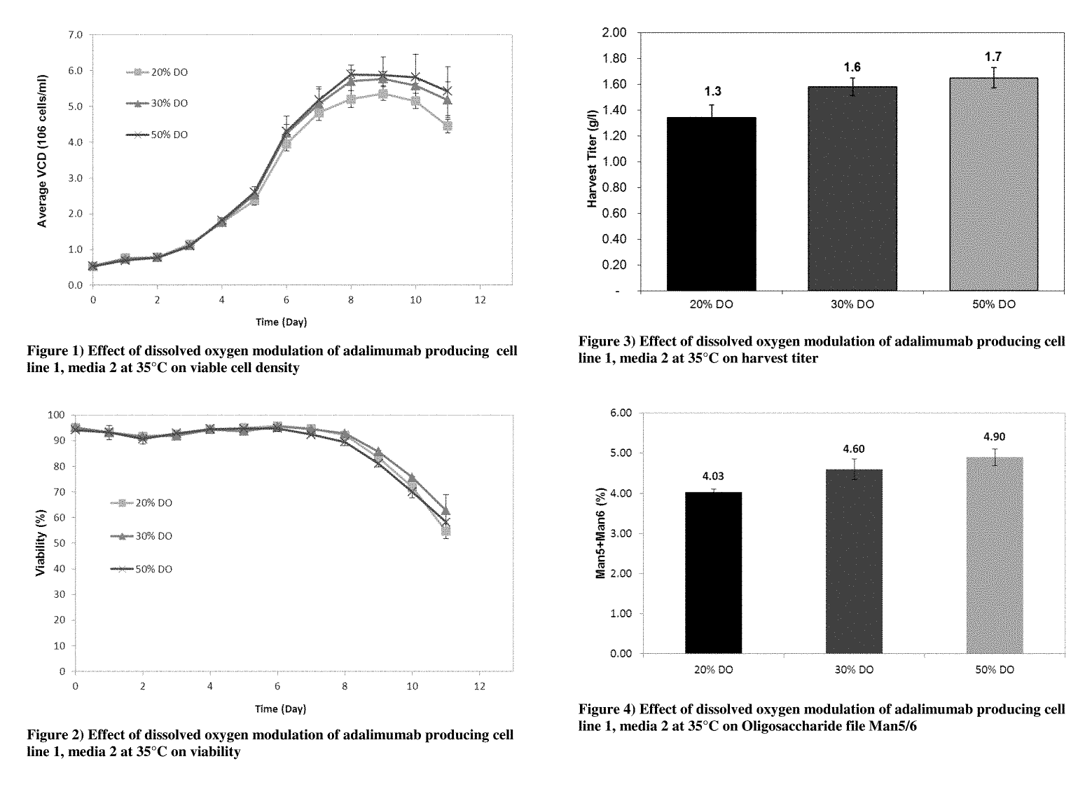

The adalimumab producing cell line 1, in media 2 at 35° C. was evaluated for the effects of dissolved oxygen concentration on the level of oligomannose. Three different dissolved oxygen (DO) conditions were evaluated, specifically 20%, 30% and 50% dissolved oxygen. The cultures were incubated at 35° C. The cell density and viability were very comparable at different DO conditions (FIGS. 1 and 2). The cultures were harvested at the target viability of 50% for each DO condition. The harvest titer was higher at 50% DO compared to 20% DO (FIG. 3). The harvest was also taken through protein A purification before oligosaccharide analysis. The percentage of oligomannose in each of the test conditions was 4.03% (20% DO), 4.60% (30% DO), and 4.90% (50% DO), respectively (FIG. 4). The percentage of oligomannose 5 and 6 was in general higher at higher dissolved oxygen concentrations. The percentage of oligomannose 5 and 6 decreased with a decrease in DO from 4.90% in the 50% DO condition to 4.03% in the 20% DO condition, for a total reduction of 0.87%.

### Example 2: The Effect of Dissolved Oxygen on the Level of Oligomannose for Cell Line 1 in Media 2 at 33° C.

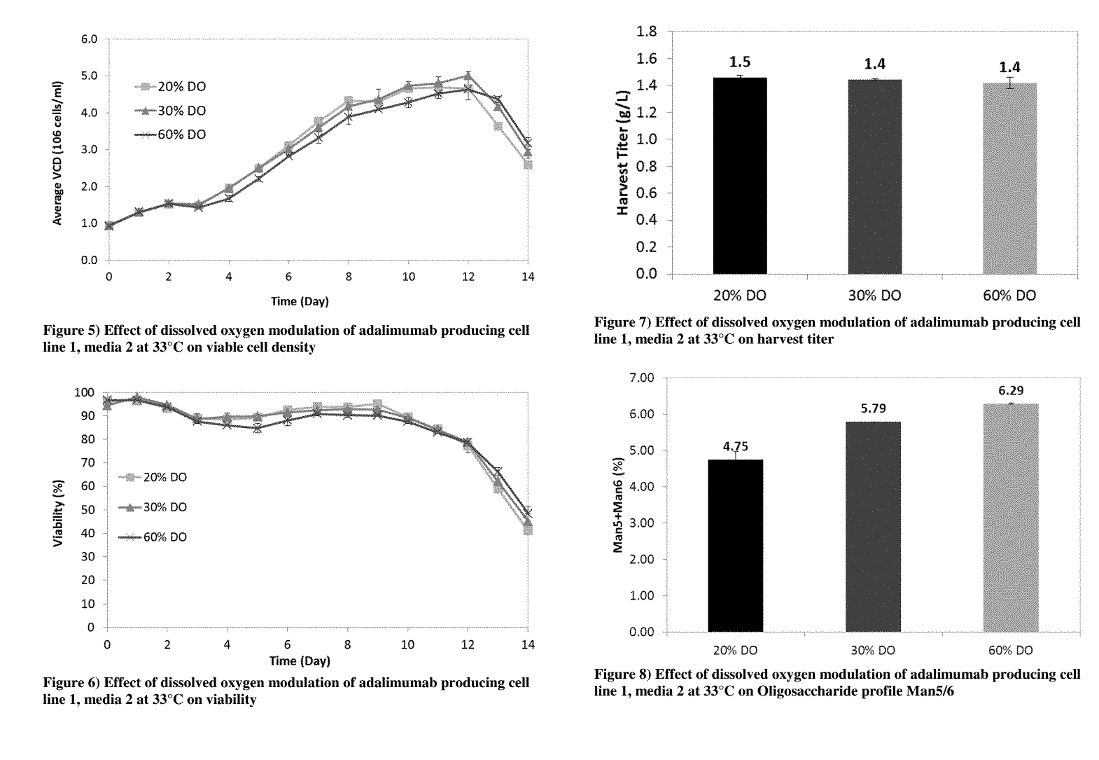

The adalimumab producing cell line 1, in media 2 at 33° C. was evaluated for the effects of dissolved oxygen concentration on the level of oligomannose. Three different dissolved oxygen (DO) conditions were evaluated, specifically 20%, 30% and 60% dissolved oxygen. The cultures were incubated at 33° C. The cell density, viability and product titer were very comparable at different DO conditions (FIGS. 5, 6 and 7). The percentage of oligomannose in each of the test conditions was 4.75% (20% DO), 5.79% (30% DO), and 6.29% (60% DO), respectively (FIG. 8). The percentage of oligomannose was in general lower at lower dissolved oxygen concentrations. The percentage of oligomannose decreased with decrease in DO from 6.29% in the 60% DO condition to 4.75% in the 20% DO condition, for a total reduction of 1.54%.

### Example 3: The Effect of Dissolved Oxygen on the Level of Oligomannose for Cell Line 1 in Media 1 at 35° C.

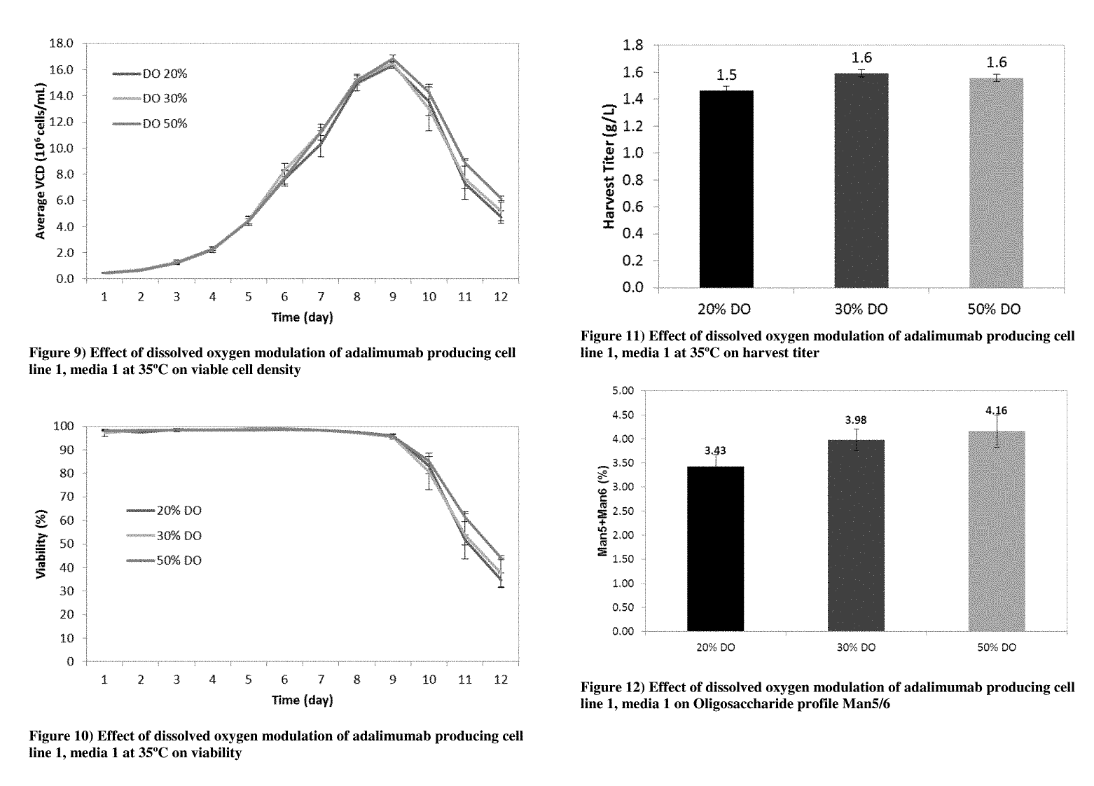

The adalimumab producing cell line 1, in media 1 at 35° C. was evaluated for the effects of dissolved oxygen concentration on the level of oligomannose. Three different dissolved (DO) conditions were evaluated, specifically 20%, 30% and 50% dissolved oxygen. The cultures were incubated at 35° C. The cell density and viability were very comparable at different DO conditions (FIGS. 9 and 10). The cultures were harvested at the target viability of 40% for each condition. The harvest titer was higher at 30% and 50% DO compared to 20% DO (FIG. 11). The harvest was also taken through protein A purification before oligosaccharide analysis. The percentage of oligomannose in each of the test conditions was 3.43% (20% DO), 3.98% (30% DO), and 4.16% (50% DO), respectively (FIG. 12). The percentage of oligomannose was in general lower at lower dissolved oxygen concentrations. The percentage of oligomannose decreased with decrease in DO from 4.16% in the 50% DO condition to 3.43% in the 20% DO condition, for a total reduction of 0.73%.

### Example 4: The Effect of Dissolved Oxygen on the Level of Oligomannose for Cell Line 3 in Media 1 at 33° C., 34° C., 35° C. and 36° C.

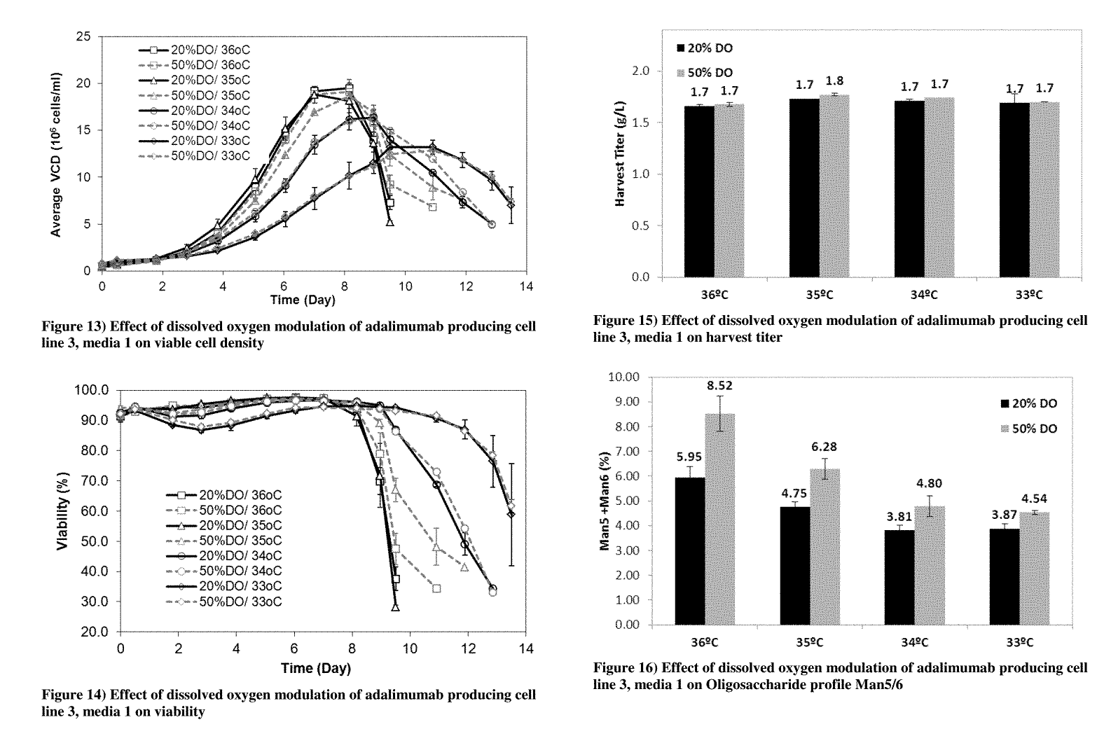

The adalimumab producing cell line 3, in media 1 at 33° C., 34° C., 35° C. and 36° C. was evaluated for the effects of dissolved oxygen concentration on the level of oligomannose. The study was performed at four different temperature levels (33° C., 34° C., 35° C. and 36° C.) with two different dissolved oxygen (DO) conditions (20% DO and 50% DO). In general, the cell growth at different dissolved oxygen levels was similar, except at 35° C., in which the cell density was lower at 50% DO (FIG. 13). The cultures were harvested either on day 10 or at ˜50% viability (FIG. 14). The titer at ˜50% viability was comparable at different DO conditions (FIG. 15). The percentage of oligomannose was in general lower at lower dissolved oxygen at each tested temperature condition (FIG. 16). On day 10, the percentage of oligomannose decreased with a decrease in DO at 36° C. from 8.52% in the 50% DO condition to 5.95% in the 20% DO condition, for a total reduction of 2.57%; the percentage of oligomannose decreased with a decrease in DO at 35° C. from 6.28% in the 50% DO condition to 4.75% in the 20% DO condition, for a total reduction of 1.53%; the percentage of oligomannose decreased with a decrease in DO at 34° C. from 4.80% in the 50% DO condition to 3.81% in the 20% DO condition, for a total reduction of 0.99%; and the percentage of oligomannose decreased with a decrease in DO at 33° C. from 4.54% in the 50% DO condition to 3.87% in the 20% DO condition, for a total reduction of 0.67%. In summary, at all tested temperature conditions, the percentage of oligomannose was lower at lower dissolved oxygen concentrations.

### Example 5: The Effect of Dissolved Oxygen on the Level of Oligomannose for the mAB2 Cell Line in Media 1 at 35° C.

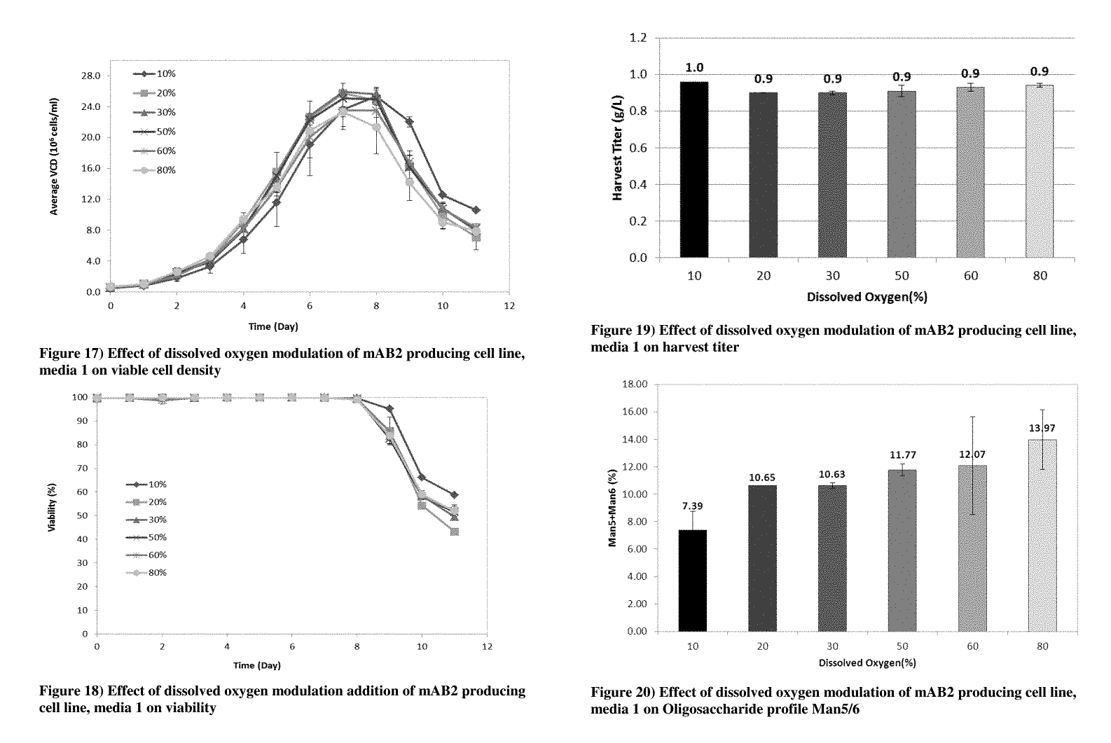

The mAB2 producing cell line, in media 1 at 35° C. was evaluated for the effects of dissolved oxygen concentration on the level of oligomannose. Six different dissolved oxygen (DO) conditions were evaluated, specifically 10%, 20%, 30%, 50%, 60% and 80% dissolved oxygen. The cultures were incubated at 35° C. In general, the cell density, viability and titer at different dissolved oxygen levels were comparable (FIGS. 17, 18 and 19). The percentage of oligomannose in each of the test conditions was estimated to be 7.39% (10% DO), 10.65% (20% DO), 10.63% (30% DO), 11.77% (50% DO), 12.07% (60% DO) and 13.97% (80% DO), respectively (FIG. 20). The percentage of oligomannose was in general lower at lower dissolved oxygen. The percentage of oligomannose decreased with decrease in DO from 13.97% in the 80% DO condition to 7.39% in the 10% DO condition, for a total reduction of 6.58%.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, GenBank Accession Numbers, and protocols that may be cited throughout this application, the disclosures of which are incorporated herein by reference in their entireties for all purposes. The contents of all cited references, including literature references, issued patents, and published patent applications, as cited throughout this application are hereby expressly incorporated herein by reference. It should further be understood that the contents of all the figures and tables attached hereto are expressly incorporated herein by reference. The entire contents of the following applications are also expressly incorporated herein by reference: International Application Serial No. PCT/US2014/061279, entitled “STABLE SOLID PROTEIN COMPOSITIONS AND METHODS OF MAKING SAME”, Attorney Docket Number 117813-31020, filed on Oct. 18, 2014; U.S. patent application Ser. No. 14/077,576, entitled “LOW ACIDIC SPECIES COMPOSITIONS AND METHODS FOR PRODUCING THE SAME USING DISPLACEMENT CHROMATOGRAPHY”, Attorney Docket Number 117813-73603, filed on Nov. 12, 2013; U.S. patent application Ser. No. 14/206,296, entitled “MUTATED ANTI-TNFa ANTIBODIES AND METHODS OF THEIR USE”, Attorney Docket Number 117813-73803, filed on Mar. 12, 2014; U.S. patent application Ser. No. 14/077,988, entitled “MODULATED LYSINE VARIANT SPECIES AND METHODS FOR PRODUCING AND USING THE SAME”, Attorney Docket Number 117813-74102, filed on Nov. 12, 2013; U.S. patent application Ser. No. 14/077,574, entitled “PURIFICATION OF PROTEINS USING HYDROPHOBIC INTERACTION CHROMATOGRAPHY”, Attorney Docket Number 117813-74302, filed on Nov. 12, 2013; and U.S. patent application Ser. No. 14/077,871, entitled “LOW ACIDIC SPECIES COMPOSITIONS AND METHODS FOR PRODUCING AND USING THE SAME”, Attorney Docket Number 117813-73902, filed on Nov. 12, 2013; U.S. patent application Ser. No. 14/209,821, entitled “METHODS FOR MODULATING PROTEIN GLYCOSYLATION PROFILES OF RECOMBINANT PROTEIN THERAPEUTICS USING MONOSACCHARIDES AND OLIGOSACCHARIDES”, Attorney Docket Number 117813-74802, filed on Mar. 13, 2014; International Application Serial No. PCT/US2015/038819, entitled “METHODS FOR MODULATING PROTEIN GLYCOSYLATION PROFILES OF RECOMBINANT PROTEIN THERAPEUTICS USING COBALT”, Attorney Docket Number 117813-76620, filed on Jul. 1, 2015; U.S. patent application Ser. No. 13/457,020, entitled “METHODS FOR CONTROLLING THE GALACTOSYLATION PROFILE OF RECOMBINANTLY-EXPRESSED PROTEINS”, Attorney Docket Number 117813-76402, filed Apr. 26, 2012; International Application Serial No. PCT/US2015/038819, entitled “METHODS FOR MODULATING PROTEIN GLYCOSYLATION PROFILES OF RECOMBINANT PROTEIN THERAPEUTICS USING COBALT”, Attorney Docket Number 117813-76620, filed on Jul. 1, 2015, U.S. patent application Ser. No. 14/795,838, entitled “METHODS FOR MODULATING THE GLYCOSYLATION PROFILE OF RECOMBINANT PROTEINS USING SUGAR”, Attorney Docket Number 117813-77102, filed on Jul. 9, 2015.

1. A method of producing a composition comprising adalimumab with a modulated glycosylation profile, said method comprising:
culturing a host cell expressing said adalimumab in cell culture media supplemented with dissolved oxygen, thereby producing said composition comprising said adalimumab with a modulated glycosylation profile as compared to a control,
wherein said control is a composition comprising said adalimumab produced by culturing a host cell expressing said adalimumab in cell culture media which is not supplemented with said dissolved oxygen.

2. The method of claim 1, further comprising purifying said composition comprising said adalimumab with a modulated glycosylation profile.

3-7. (canceled)

8. The method of claim 1, wherein the cell culture media is supplemented with a sufficient amount of the dissolved oxygen to achieve a dissolved oxygen concentration selected from the group consisting of about 10%, about 20%, about 30%, about 40%, about 50%, about 60%, about 70%, and about 80%.

9-16. (canceled)

17. The method of claim 1, wherein the modulated glycosylation profile of the adalimumab comprises modulation of a galactosylation level, a mannosylated N-glycan level or an agalactosyl N-glycan level in said adalimumab.

18-21. (canceled)

22. The method of claim 17, wherein the modulation of the mannosylated N-glycan level comprises a decrease in the mannosylation level of said adalimumab.

23. The method of claim 22, wherein the decrease in the mannosylation level comprises a decrease in the level of Man 5 glycan, Man 6 glycan, Man 7 glycan, Man 8 glycan and/or Man 9 glycan.

24. The method of claim 23, wherein the decrease in the level of Man 5 glycan, Man 6 glycan, Man 7 glycan, Man 8 glycan and/or Man 9 glycan is a decrease of about 0.1%, 1%, 1.2%, 1.5%, 2%, 2.2%, 2.5%, 3%, 3.2%, 3.5%, 4%, 4.2%, 4.5%, 5%, 10%, 15%, 20%, or 25%.

25. The method of claim 23, wherein the decrease in the level of Man 5 glycan, Man 6 glycan, Man 7 glycan, Man 8 glycan and/or Man 9 glycan is a decrease of about 2.55%.

26. (canceled)

27. The method of claim 1, wherein said host cell is a CHO cell.

28-36. (canceled)

37. A composition comprising a cell culture media comprising dissolved oxygen.

38. The composition of claim 37, wherein the cell culture media comprises a dissolved oxygen concentration selected from the group consisting of about 10%, about 20%, about 30%, about 40%, about 50%, about 60%, about 70%, and about 80%.

39. A pharmaceutical composition comprising the composition produced by the method of claim 1 and a pharmaceutically acceptable carrier.

40. A composition comprising adalimumab with a modulated glycosylation profile produced by the method of claim 1.

41-42. (canceled)

43. A composition comprising adalimumab, wherein said adalimumab comprises a 0.1-15% decrease in the level of mannosylated N-glycans as compared to a control,
wherein said control is a composition comprising adalimumab produced by culturing a host cell expressing adalimumab in cell culture media which is not supplemented with dissolved oxygen.

44. The composition of claim 43, wherein the decrease in the level of mannosylated N-glycans as compared to a control is a decrease of about 2.55%.

45-58. (canceled)


SEQUENCE LISTING (12 sequences)

SEQ ID NO: 1 - protein, 107 aa, Artificial Sequence [sequence omitted]

SEQ ID NO: 2 - protein, 121 aa, Artificial Sequence [sequence omitted]

SEQ ID NO: 3 - protein, 9 aa, Artificial Sequence
  Description of Artificial Sequence Synthetic peptide
  Adalimumab light chain variable region CDR3
  MOD_RES (9)..(9): Thr or Ala
QRYNRAPYX

SEQ ID NO: 4 - protein, 12 aa, Artificial Sequence
  Description of Artificial Sequence Synthetic peptide
  Adalimumab heavy chain variable region CDR3
  MOD_RES (12)..(12): Tyr or Asn
VSYLSTASSLDX

SEQ ID NO: 5 - protein, 7 aa, Artificial Sequence
  Description of Artificial Sequence Synthetic peptide
  Adalimumab light chain variable region CDR2
AASTLQS

SEQ ID NO: 6 - protein, 17 aa, Artificial Sequence
  Description of Artificial Sequence Synthetic peptide
  Adalimumab heavy chain variable region CDR2
AITWNSGHIDYADSVEG

SEQ ID NO: 7 - protein, 11 aa, Artificial Sequence
  Description of Artificial Sequence Synthetic peptide
  Adalimumab light chain variable region CDR1
RASQGIRNYLA

SEQ ID NO: 8 - protein, 5 aa, Artificial Sequence
  Description of Artificial Sequence Synthetic peptide
  Adalimumab heavy chain variable region CDR1
DYAMH

SEQ ID NO: 9 - DNA, 321 bp, Artificial Sequence
  Description of Artificial Sequence Synthetic polynucleotide
  Adalimumab light chain variable region
gacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaa

SEQ ID NO: 10 - DNA, 363 bp, Artificial Sequence
  Description of Artificial Sequence Synthetic polynucleotide
  Adalimumab heavy chain variable region
gaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagt

SEQ ID NO: 11 - protein, 214 aa, Artificial Sequence
  Description of Artificial Sequence Synthetic polypeptide
  Adalimumab light chain
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 451 aa, Artificial Sequence
  Description of Artificial Sequence Synthetic polypeptide
  Adalimumab heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK